(19) United States
(12) Patent Application Publication
(10) Pub. No.: US 2025/0127476 A1
(43) Pub. Date: Apr. 24, 2025

(54) METHODS, SYSTEMS, AND MEDIUMS FOR SCANNING

(71) Applicant: SHANGHAI UNITED IMAGING HEALTHCARE CO., LTD., Shanghai (CN)

(72) Inventors: Biao SUN, Shanghai (CN); Chenghang HAN, Shanghai (CN); An ZHAO, Shanghai (CN)

(73) Assignee: SHANGHAI UNITED IMAGING HEALTHCARE CO., LTD., Shanghai (CN)

(21) Appl. No.: 19/000,781

(22) Filed: Dec. 24, 2024

Related U.S. Application Data
(63) Continuation of application No. PCT/CN2023/121709, filed on Sep. 26, 2023.

(30) Foreign Application Priority Data
Sep. 26, 2022 (CN) 202211175599.3
Dec. 30, 2022 (CN) 202211738750.X

(51) Int. Cl. A61B 6/00; A61B 6/10; A61B 6/46; A61B 6/50

(52) U.S. Cl. CPC A61B 6/542; A61B 6/102; A61B 6/466; A61B 6/467; A61B 6/481; A61B 6/507; A61B 6/5217; A61B 6/563

(57) ABSTRACT

A method and system for scanning are provided. The method may include obtaining one or more images of a target subject acquired by an imaging device; determining a three-dimensional (3D) geometric model of the target subject based on the one or more images; obtaining an anatomical structure model of at least a portion of the target subject; obtaining a combination model by combining the 3D geometric model of the target subject and the anatomical structure model of at least a portion of the target subject; and determining one or more scanning parameters of the target subject based on the combination model.

### CROSS-REFERENCE TO RELATED APPLICATIONS

This application is a Continuation of International Application No. PCT/CN2023/121709, filed on Sep. 26, 2023, which claims priority to the Chinese Patent Application No. 202211738750.X, filed on Dec. 30, 2022, and Chinese Patent Application No. 202211175599.3, filed on Sep. 26, 2022, the contents of each of which are hereby incorporated by reference.

### TECHNICAL FIELD

The present disclosure relates to a medical imaging technology field, in particular, relates to systems, methods, and mediums for scanning.

### BACKGROUND

In a routine interventional procedure, if users need to perform angiography on a portion of a patient (e.g., lower limb blood vessels, coronary arteries, etc.), fussy operations may be often performed. For example, a specific process of the traditional stepping acquisition may typically involve raising a bed, moving the bed towards the head of the patient, adjusting a detector vertically, and maximizing a SID (i.e., an identification of an X-ray device system); locating an ending point of localization imaging and recording a location of the ending point under X-ray fluoroscopy. The above manners may lead to problems such as repeated operations by the users, a relatively high radiation exposure, a relatively long preparation time for acquisition during the process, etc.

Therefore, it is desirable to provide a system and method for scanning that can solve shortcomings of traditional manners such as complex operation, a long preparation time, and low acquisition efficiency.

### SUMMARY

One aspect of embodiments of the present disclosure may provide a system. The system may include at least one storage medium including a set of instructions; at least one processor in communication with the at least one storage medium, wherein when executing the set of instructions, the at least one processor is directed to cause the system to perform operations including: obtaining one or more images of a target subject acquired by an imaging device; determining a three-dimensional (3D) geometric model of the target subject based on the one or more images; obtaining an anatomical structure model of at least a portion of the target subject; obtaining a combination model by combining the 3D geometric model of the target subject and the anatomical structure model of at least a portion of the target subject; and determining one or more scanning parameters of the target subject based on the combination model.

In some embodiments, the determining one or more scanning parameters of the target subject based on the combination model may include displaying the combination model on a user interface; obtaining a first input of a user via the user interface generated according to the combination model; and determining the one or more scanning parameters based on the first input of the user.

In some embodiments, the operations may further include: obtaining a second input of the user via the user interface generated according to the combination model; and adjusting the one or more scanning parameters based on the second input of the user.

In some embodiments, the one or more scanning parameters may include a scanning range defined by a starting point and an ending point, the operations may further include: in a first image acquisition stage before a target portion of the target subject is injected with a contrast agent, causing a second imaging device to arrive at the starting point and/or the ending point; and obtaining a first image by causing the second imaging device to perform a scan.

In some embodiments, the operations may further include: in a second image acquisition stage after the target portion of the target subject is injected with the contrast agent, determining a blood flow velocity of the target portion; adjusting a moving speed of the second imaging device based on the blood flow velocity; and obtaining a second image by causing the second imaging device to perform a scan.

In some embodiments, the operations may further include obtaining a target image of the target portion of the target subject based on the first image and the second image.

In some embodiments, the determining a blood flow velocity of the target portion may include: obtaining a third image of the target portion of the target subject; and determining the blood flow velocity based on the third image.

In some embodiments, the determining a blood flow velocity of the target portion may include: determining a moving distance and a duration corresponding to the moving distance of the contrast agent in the target subject; and determining the blood flow velocity based on the moving distance and the duration corresponding to the moving distance of the contrast agent in the target subject.

In some embodiments, the operations may further include causing a second imaging device to perform multiple rounds of scans on the target portion of the target subject based on the one or more scanning parameters.

In some embodiments, the target portion includes the heart of the target subject, and the causing a second imaging device to perform multiple rounds of scans on the target portion of the target subject based on the one or more scanning parameters includes: causing the second imaging device to perform a rotation scan on the heart, the rotation scan including the multiple rounds of scans, each of the multiple rounds of scans corresponding to a rotation angle.

In some embodiments, the target portion includes a leg of the target subject, and the causing a second imaging device to perform multiple rounds of scans on the target portion of the target subject based on the one or more scanning parameters includes: causing the second imaging device to perform a stepping scan on the leg, the stepping scan including the multiple rounds of scans, each of the multiple rounds of scans corresponding to one bed position.

In some embodiments, the imaging device may include at least one of a visible light sensor, an infrared sensor, or a radar sensor.

In some embodiments, the anatomical structure model of the at least a portion of the target subject may be acquired based on one or more images acquired by a second imaging device.

In some embodiments, the one or more scanning parameters include a scanning range, the operations may further include: causing one or more components of a second imaging device to move to a target position, at the target position, the scanning range of the target subject being located at an isocenter of the second imaging device; and causing the second imaging device to perform a scan on the target subject.

In some embodiments, the one or more scanning parameters may include a rotation angle of one or more components of a second imaging device for performing a scan on the target subject, and the determining one or more scanning parameters of the target subject based on the combination model includes: determining the rotation angle by adjusting an initial rotation angle.

In some embodiments, the adjusting an initial rotation angle may include: determining a distance between the target subject and the one or more components of the second imaging device based on the combination model; and adjusting the initial rotation angle based on the distance.

In some embodiments, the adjusting the initial rotation angle based on the distance may include in response to determining that the distance is less than a distance threshold, adjusting the initial rotation angle to obtain the rotation angle, a distance between the target subject and the one or more components of the second imaging device after the initial rotation angle is adjusted exceeds the distance threshold.

In some embodiments, the one or more scanning parameters includes a scanning route of one or more components of a second imaging device for performing a scan on the target subject, the scanning route indicating a moving trajectory of the one or more components of the second imaging device, and the operations may further include predicting whether a collision involves in the scan based on the scanning route; in response to determining that the collision involves in the scan, adjusting the scanning route; and causing the second imaging device to perform the scan based on the adjusted scanning route.

In some embodiments, the operations may further include in response to determining that the collision involves in the scan, generating a reminder for the collision, the reminder may include at least one of collision alarm, a position of the collision, or the adjustment of the scanning route.

In some embodiments, the determining one or more scanning parameters of the target subject based on the combination model may include: obtaining a trained machine learning model; and determining the one or more scanning parameters of the target subject based on the combination model and the trained machine learning model.

Another aspect of embodiments of the present disclosure may provide a method. The method may include obtaining one or more images of a target subject acquired by an imaging device; determining a three-dimensional (3D) geometric model of the target subject based on the one or more images; obtaining an anatomical structure model of at least a portion of the target subject; obtaining a combination model by combining the 3D geometric model of the target subject and the anatomical structure model of at least a portion of the target subject; and determining one or more scanning parameters of the target subject based on the combination model.

Another aspect of embodiments of the present disclosure may provide a non-transitory computer readable medium. The non-transitory computer readable medium may comprise a set of instructions, wherein when executed by at least one processor, the set of instructions direct the at least one processor to perform acts of: obtaining one or more images of a target subject acquired by an imaging device; determining a three-dimensional (3D) geometric model of the target subject based on the one or more images; obtaining an anatomical structure model of at least a portion of the target subject; obtaining a combination model by combining the 3D geometric model of the target subject and the anatomical structure model of at least a portion of the target subject; and determining one or more scanning parameters of the target subject based on the combination model.

### BRIEF DESCRIPTION OF THE DRAWINGS

The present disclosure is further describable in terms of exemplary embodiments. These exemplary embodiments are describable in detail with reference to the drawings. These embodiments are non-limiting exemplary embodiments, in which like reference numerals represent similar structures throughout the several views of the drawings, and wherein:

FIG. 1 is a schematic diagram illustrating an exemplary application scenario of a system for scanning according to some embodiments of the present disclosure;

FIG. 2 is a flowchart illustrating an exemplary process for imaging according to some embodiments of the present disclosure;

FIG. 3 is a flowchart illustrating an exemplary process 300 for a stepping acquisition according to some embodiments of the present disclosure;

FIG. 4 is a flowchart illustrating an exemplary process 350 for a stepping acquisition according to some embodiments of the present disclosure;

FIG. 5 is a schematic diagram illustrating another exemplary process for a stepping acquisition of a digital subtraction angiography (DSA) device according to some embodiments of the present disclosure;

FIG. 6 is a schematic diagram illustrating another exemplary process for a stepping acquisition of a digital subtraction angiography (DSA) device according to some embodiments of the present disclosure;

FIG. 7 is a schematic diagram illustrating an exemplary process for a stepping acquisition of a digital subtraction angiography (DSA) device according to some embodiments of the present disclosure;

FIG. 8 is a flowchart 1 illustrating an exemplary process for controlling a scan of a portion of a target subject according to some embodiments of the present disclosure;

FIG. 9 is a flowchart illustrating an exemplary process for controlling a scan of a portion of a target subject in operation 801 according to some embodiments of the present disclosure;

FIG. 10 is a flowchart illustrating an exemplary process for controlling a scan of a portion of a target subject according to some embodiments of the present disclosure;

FIG. 11 is a flowchart 3 illustrating an exemplary process for controlling a scan of a portion of a target subject according to some embodiments of the present disclosure;

FIG. 12 is a schematic diagram illustrating an exemplary rotating scanning process of a second imaging device according to some embodiments of the present disclosure;

FIG. 13 is a schematic diagram illustrating an exemplary module of a scanning system according to some embodiments of the present disclosure;

FIG. 14 is a schematic diagram illustrating an exemplary module of a control system for scanning a portion of a target subject according to some embodiments of the present disclosure; and

FIG. 15 is a schematic diagram illustrating an exemplary structure of an electronic device according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been describable at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assemblies of different levels in ascending order. However, the terms may be displaced by other expressions if they achieve the same purpose.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

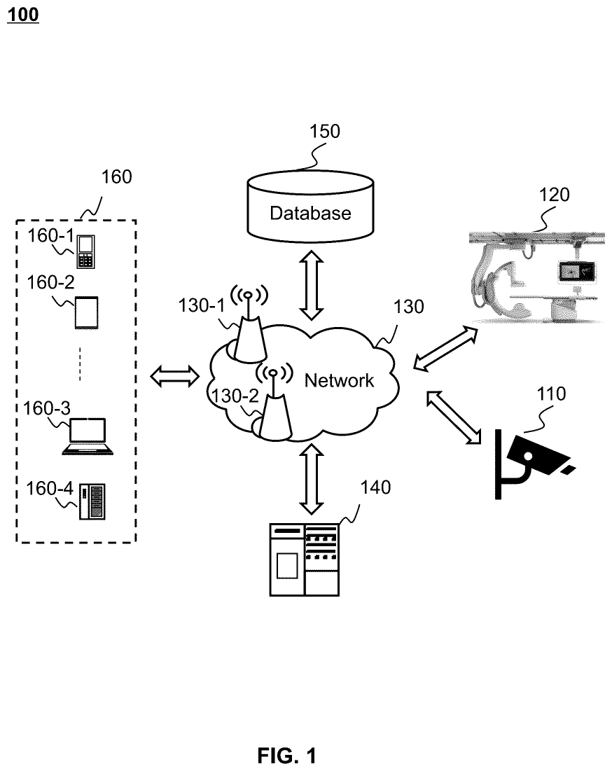

FIG. 1 is a schematic diagram illustrating an exemplary application scenario of a system for scanning according to some embodiments of the present disclosure.

As shown in FIG. 1, a scanning system 100 may include a first imaging device 110 (also referred to as an imaging device), a second imaging device 120, a network 130, a processing device 140, a storage device 150, and a terminal device 160. A plurality of components in the scanning system 100 may communicate directly or through the network 130.

The first imaging device 110 refers to a device used to capture one or more optical images of a target subject. The first imaging device 110 may be a real-time imaging device such as a camera (e.g., a digital color camera, 3D camera, etc.), a red, green, and blue (RGB) sensor, a depth sensor, an RGB depth (RGB-D) sensor, a thermal sensor (e.g., an infrared (FIR) or near-infrared (NIR) sensor), a radar sensor, and/or other types of image capture circuits configured to generate images (e.g., 2D images or photos) of a human, an object, a scene, or the like, or a combination thereof. The first imaging device 110 may acquire optical images that are used to reconstruct a three-dimension (3D) geometric model of the target subject by obtaining real-time optical image information.

In some embodiments, the target subject may include a human, an animal, or the like. A target portion of the target subject may be an entire target subject or a portion of the target subject. For example, the target portion of the target subject may be the head, the chest, the abdomen, the heart, the liver, an upper limb, a lower limb, or any combination thereof.

In some embodiments, the second imaging device 120 refers to a medical imaging device configured to execute medical collection functions, and perform automatic region selection, route planning, and automatic scanning t. the second imaging device 120 may be a medical imaging device. The second imaging device 120 may reproduce a structure of the target subject into one or more specific medical images by utilizing different media. For example, the second imaging device 120 may be a digital subtraction angiography (DSA) device (the digital subtraction angiography (DSA) device, including the C-arm and/or rack, etc.), a computed radiography (CR) system, a digital radiography (DR) system, a computed tomography (CT) device, an ultrasound imaging device, a fluoroscopy imaging device, a magnetic resonance imaging (MRI) device, or the like, or any combination thereof. For example, when the second imaging device 120 is a DSA device, the DSA device may be used to acquire one or more angiography images of the patient before and/or during intervention surgery. For example, when the second imaging device 120 is a CT device, the CT device may be used to acquire one or more CT images of the patient (e.g., computed tomography blood vessel images, CT angiography (CTA) images) before and/or during intervention surgery. In some embodiments, the second imaging device 120 may be another scanning device used to generate medical images.

In some embodiments, the first imaging device 110 and the second imaging device 120 may obtain images of the target object and medical images, respectively, and send the obtained images to the processing device 140. In some embodiments, the medical images may include scanning images, for example, a CTA image, a DSA image, or the like or a combination thereof. In some embodiments, the images obtained by the first imaging device 110 and the second imaging device 120 may be stored in the storage device 150. In some embodiments, the first imaging device 110 and the second imaging device 120 may receive imaging instructions sent from the user terminal (not shown in the figure) or the processing device 140 through the network 130 and may send imaging results to the processing device 140 or the storage device 150. In some embodiments, one or more components (e.g., the processing device 140, the storage device 150) in the scanning system 100 may be included within the first imaging device 110.

The network 130 may include any suitable network that facilitates the exchange of information and/or data by the scanning system 100. In some embodiments, the one or more other components of the scanning system 100 (e.g., the first imaging device 110, the second imaging device 120, the processing device 140, the storage device 150, etc.) may exchange information and/or data with each other through the network 130. For example, the processing device 140 may obtain image data from the first imaging device 110 and the second imaging device 120 through the network 130. For example, the processing device 140 may obtain instructions from the processing device 140 through the network 130.

The processing device 140 may process data and/or information obtained from the first imaging device 110, the second imaging device 120, and/or the storage device 150. For example, the processing device 140 may obtain one or more images from the first imaging device 110, and determine a three-dimensional (3D) geometric model of the target subject based on one or more images. In some embodiments, the processing device 140 may be local or remote. For example, the processing device 140 may access information and/or data stored in the first imaging device 110, the second imaging device 120, and/or the storage device 150 through the network 130. For example, the processing device 140 may be directly connected with the first imaging device 110, the second imaging device 120, and/or the storage device 150 to access the stored information and/or data.

The storage device 150 may store data, instructions, and/or other information. In some embodiments, the storage device 150 may store data obtained from the terminal device 160 and/or the processing device 140. In some embodiments, the storage device 150 may store data and/or instructions executed or used by the processing device 140 to execute the exemplary processes described in the present disclosure. In some embodiments, the storage device 150 may be parts of the processing device 140.

The terminal device 160 may control other components in the scanning system 100 and/or display various information and/or data. The terminal device 160 may include a user interface, which may display a combination model of the target subject. The user interface may also be configured to achieve an interaction between the user and the scanning system and/or between the target subject and the scanning system. In some embodiments, the terminal device 160 may control the second imaging device 120 to perform various operations through the instructions, such as scanning and acquiring the medical images, sending the medical images to the processing device 140, or the like. The terminal device 160 may obtain or send the information and/or data to other components of the scanning system 100. In some embodiments, the terminal device 160 may display the obtained information and/or data to the user (e.g., a doctor, etc.). In some embodiments, the terminal device 160 may include a mobile device 160-1, a tablet computer 160-2, a laptop computer 160-3, a desktop computer 160-4, or any combination thereof. In some embodiments, the terminal device 160 may be parts of the processing device 140 and/or integrated with the processing device 140.

The above description of the scanning system 100 is only illustrative. In some embodiments, the scanning system 100 may include one or more other components (e.g., the terminal that enable the user interaction), or may not include one or more of the components described above. The two or more components may also be integrated into a component.

It should be noted that the above description of the scanning system 100 and its modules is for convenience only and cannot limit the present disclosure to the scope of the cited embodiments. It can be understood that technical personnel in this field, after understanding the principle of the system, may arbitrarily combine various modules or form subsystems to connect with other modules without deviating from this principle. In some embodiments, the storage device and processing device disclosed in FIG. 1 may be different modules within a system or a module that implements the functions of two or more modules mentioned above. For example, each module can share a common storage module, and each module can also have its storage module. Such deformations are within the protection scope of the present disclosure.

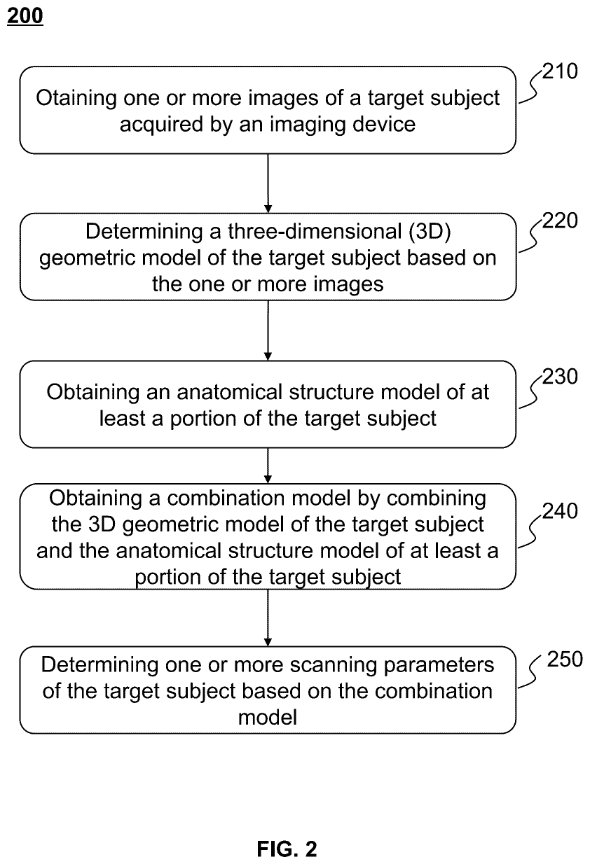

FIG. 2 is a flowchart illustrating an exemplary process for imaging according to some embodiments of the present disclosure.

In some embodiments, process 200 may be executed by the processing device 140. In some embodiments, process 200 may be executed by the scanning system. In some embodiments, process 200 may be executed by the electronic device. As shown in FIG. 2, the process 200 may include the following operations:

In 210, one or more images of a target subject acquired by an imaging device (also referred to as a first imaging device) may be obtained.

More descriptions of the first imaging device and the target subject may be found in FIG. 1 and related descriptions.

In some embodiments, the imaging device may include at least one of a visible light sensor, an infrared sensor, a radar sensor, or the like, or a combination thereof.

Correspondingly, depending on the types of the used sensor or image capture circuit, the photos of the target subject generated by the imaging device may include one or more images of the target subject captured by a visible light sensor, one or more thermal images of the target subject generated by an infrared sensor, one or more radar images of the target subject generated by a radar sensor, or the like, or a combination thereof.

In some embodiments of the present disclosure, various types of images such as photos, thermal images, and radar images may be captured by the visible light sensor, the infrared sensor, or the radar sensor, which is beneficial for establishing a three-dimensional (3D) geometric model of the target subject, improving the accuracy of the 3D geometric model.

The one or more images may include a 2D image, a 3D image, or the like. The one or more images of the target subject acquired by the imaging device may be a full body image of the target subject or a local image of the target subject, such as lower limb images, chest images, abdominal images, or the like.

In some embodiments, the processing device may detect the target subject by using a real-time or periodic continuous acquisition through the imaging device. For example, the processing device may detect the target subject by using a real-time or interval acquisition through the imaging device. Through the real-time acquisition, even if the target subject moves or changes in pose, the image of the target subject may still reflect a current pose of the target subject, or the like. In some embodiments, the duration of data acquisition by the imaging device may be preset in the system.

In 220, a three-dimensional (3D) geometric model of the target subject may be determined based on the one or more images.

The 3D geometric model refers to a model that represents an external structure of the target subject. For example, the 3D geometric model represents a 3D model of an entire body or local external structure of the target subject. For example, the 3D geometric model indicates a contour and/or pose of the target subject during the acquisition of the one or more images. The pose of the target subject reflects one or more of position, posture, shape, size, or the like.

In some embodiments, the 3D geometric model of the target subject may be represented by one or more of a 3D mesh, a 3D contour, or the like, to indicate the pose, the body shape, or other features of the target subject.

The processing device may determine the 3D geometric model of the target subject in various ways. In some embodiments, the processing device may generate or construct the 3D geometric model based on the one or more images of the target subject through a modeling software, a neural network model, etc. The 3D geometric model may include a parametric model, a skin multiplayer linear (SMPL) model, or the like. The modeling software may include Maya, 3ds Max, or the like.

In 230, an anatomical structure model of at least a portion of the target subject may be obtained.

The anatomical structure model refers to a model of a portion or all organs or tissues related to the anatomical structure of the target subject. For example, the anatomical structure model may be a model related to myocardium, knee joint cartilage, blood vessels, or the like.

In some embodiments, the anatomical structure model of the at least a portion of the target subject may be denoted via an anatomical image (e.g., a 3D anatomical image).

For example, the target subject may include a blood vessel and the anatomical structure model may be denoted by a CTA image of the target subject. As another example, the target subject may include the heart and the anatomical structure model may be denoted by the heart image of the target subject.

In some embodiments, the anatomical structure model may be composed of multiple voxels. Each of the multiple voxels may have corresponding coordinates denoted by a coordinate system applied to the target subject or a reference object (e.g., the second imaging device, the first imaging device, etc.). Each voxel represents a portion of the target subject, and the multiple voxels corresponding to the target subject may correspond to different components of the target subject.

The anatomical structure model of at least a portion of the target subject may be obtained through various processes. For example, the processing device may obtain the anatomical structure model of the target subject from the storage device. As another example, the anatomical structure model may include a historical anatomical image obtained by the second imaging device in a historical scan of the target subject. An imaging range of the imaging device may be a detection region of the second imaging device. More descriptions of the second imaging device may be found in FIG. 1 and related descriptions.

In some embodiments, the anatomical structure model may include a reference anatomical image of a reference object. The reference object may be a biological object or a non-biological object that have an internal anatomical structure similar to the target subject. The reference anatomical image may represent a standard anatomical structure model of the target subject. In some embodiments, the standard anatomical structure model may be acquired based on multiple anatomical images of reference objects (e.g., different blood vessels). For example, the multiple anatomical images of the reference objects may be registered and averaged to obtain the standard anatomical structure model. In some embodiments, the standard anatomical structure model may be acquired based on an anatomical image of a phantom. The phantom may have an internal anatomical structure similar to the target subject.

In some embodiments, the anatomical structure model of at least a portion of the target subject may be acquired based on one or more images acquired by a second imaging device. In some embodiments, the anatomical structure model of at least a portion of the target subject may be an anatomical structure model obtained based on historical imaging devices or other imaging devices. For example, the anatomical structure model of at least a portion of the target subject maybe obtained based on historical medical images of the target subject acquired by historical imaging devices or other imaging devices in a different modality.

In some embodiments, medical images may be two-dimensional (2D) and/or three-dimensional (3D) images of the internal structure of the target subject. In the 2D image, the smallest distinguishable element may be a pixel point. In the 3D image, the smallest distinguishable element may be a voxel point. In the 3D image, images may be composed of a series of 2D slices or dimensional layers.

In some embodiments, medical images may be a series of anatomical structure images or electronic data reflecting the anatomical structure. For example, a series of angiography images may be images of different cross-sections of a certain anatomical structure.

In some embodiments, the processing device may construct the anatomical structure model based on one or more medical images. For example, if the scanning data of the second imaging device is a series of medical images of different cross-sections of the anatomical structure, the processing device may combine the cross-sections of the anatomical structure in space based on the contours of the anatomical structure in each cross-section and corresponding coordinates of the cross-section to synthesize the complete anatomical structure model. In some embodiments, the anatomical structure model may be constructed based on the electronic data (e.g., the scanning data). For example, if the scanning data is coordinates of different voxel points on the anatomical structure of the target subject output by the second imaging device, a process of establishing the anatomical structure model may include arranging the coordinates of the voxel points in space and synthesizing the complete anatomical structure model.

In some embodiments of the present disclosure, the one or more medical images obtained through the second imaging device can improve the accuracy of the anatomical structure model, which can align with an actual situation of the target subject better, and facilitate the subsequent acquisition of reliable scanning parameters.

In 240, a combination model may be obtained by combining the 3D geometric model of the target subject and the anatomical structure model of at least a portion of the target subject.

The combination model represents a geometric structure (e.g., pose, shape, size) of the target subject and an anatomical structure of the target subject. The combination model represents the external structure (e.g., pose, shape, size) of the target subject and the internal structure of the target subject.

In some embodiments, the processing device may generate the combination model of the target subject by combining the 3D geometric model and the anatomical structure model. The type of generated combination model may be the same or different from the type of the 3D geometric model. For example, the 3D geometric model may be a 3D mesh model that represents the external structure of the target subject, and the combination model may be a 3D mesh model that represents the external structure and the internal structure of the target subject.

For example, the 3D geometric model may be generated based on image data of the body surface of the patient. The anatomical structure model may be a historical anatomical image of a portion of the patient. The processing device may generate a combination model by combining the 3D geometric model with the anatomical structure model. The combination model may not only represent an external structure of the patient (e.g., shape, size, or posture), but also represent an internal structure of the patient.

In some embodiments, the processing device may perform one or more image processing operations (e.g., fusion operation, image registration operation, etc.) or any combination thereof to combine the 3D geometric model and the anatomical structure model. The fusion operation may include a data level (or pixel level) image fusion operation, a feature level image fusion operation, a decision level image fusion operation, or any combination thereof. The fusion operation may be performed based on an algorithm such as a maximum density projection algorithm, a multi-scale analysis algorithm, a wavelet transform algorithm, or the like. In some embodiments, the processing device may register the anatomical structure model to the 3D geometric model before the fusion operation. The processing device may register, using a registration algorithm, the anatomical structure model to the 3D geometric model in the same coordinate system as the 3D geometric model to obtain the registered anatomical structure model. The processing device may also generate a combination model by fusing the registered anatomical structure model and the 3D geometric model. Exemplary registration algorithms may include a grayscale-based registration algorithm, an image feature-based registration algorithm, or the like, or a combination thereof.

In some embodiments, the combination model may be generated before or during the scan on the target subject. Optionally, the image data of the target subject for determining the 3D geometric model may be acquired continuously or intermittently (e.g., periodically), and the combination model may be updated continuously or intermittently (e.g., periodically) based on the image data of the target subject.

In 250, one or more scanning parameters of the target subject may be determined based on the combination model.

The scanning parameters refer to parameters used by the second imaging device when scanning the target subject. For example, the scanning parameter may include a scanning region, a scanning route, a scanning angle, a rotation angle, a scanning sequence, an exposure parameter, or any combination thereof, of the second imaging device. The scanning region refers to a portion of the target subject (e.g., a specific organ or tissue) that the second imaging device needs to scan for imaging (or examination, or treatment). The scanning region may also be referred to as a scanning range. The scanning route refers to a movement trajectory of a component (e.g., a gantry, a C arm, etc.) of the second imaging device. The scanning angle refers to an angle at which the target subject is scanned. For example, the second imaging device may include a radiation source and a detector, and the scanning angle refers to an angle formed between the target subject (e.g., a coronal plane of the target subject) and a line connecting the radiation source and the detector. The rotation angle refers to an angle at which one or more components of the second imaging device rotate. The scanning sequence refers to a sequence used in magnetic resonance imaging (e.g., a spin echo sequence, a gradient echo sequence, a diffusion sequence, an inversion recovery sequence, etc.).

During the scan of the target subject using the second imaging device, a scanning region that is large enough may be needed to cover the target portion of the target subject (e.g., an organ, a lesion region on an organ), resulting in obtaining necessary information related to the target region. However, if the scanning region is much larger than the target region, harmful radiation damage may be caused to a region of the target subject outside the target region. Therefore, the processing device needs to determine a reasonable scanning region to cover the target portion of the target subject.

In some embodiments, the processing device may identify the scanning region covering a target portion of the target subject to be scanned by the second imaging device in the combination model. For example, the processing device may determine the target portion of the target subject (e.g., heart, lower limbs, etc.) that the patient needs to be further scanned or treated based on a pre-existing anatomical structure model (i.e., the anatomical structure model obtained in 230) obtained from the second imaging device. The processing device may register one or more pre-existing anatomical structure model with the 3D geometric model of the target subject generated by the imaging device (e.g., a 3D mesh of the patient), and determine a position of the target portion of the target subject on the 3D mesh. The processing device may visually indicate the determined scanning region to the target subject or medical expert by marking the scanning region on the 3D mesh.

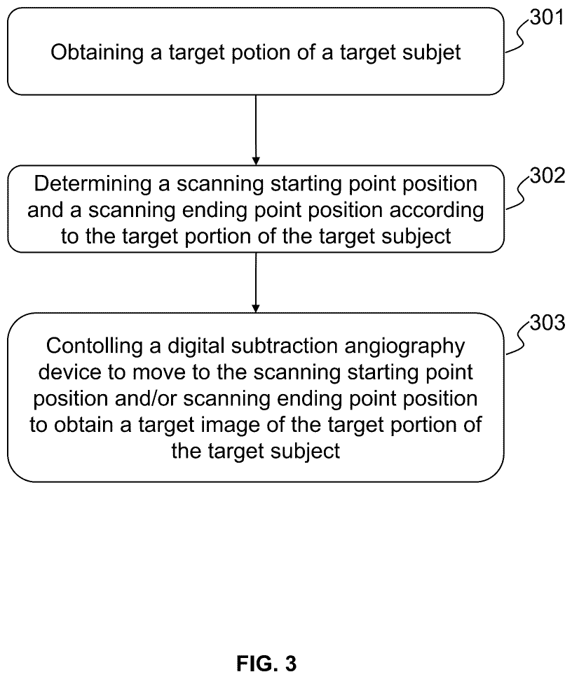

In some embodiments, the scanning region may include a scanning starting ending point position and a scanning ending point position. The scanning starting ending point position refers to the initial position at which the second imaging device starts for the scan. The scanning ending point position refers to the final position where the second imaging device ends for the scan. More descriptions of the scanning starting ending point position and the scanning ending point position may be found in FIG. 3-FIG. 7 and related descriptions.

The scanning parameters may be determined in various ways. In some embodiments, users (e.g., doctors) may manually set at least a portion of the scanning parameters (e.g., the scanning region) according to the combination model. For example, the user may input the scanning region on the combination model via the user interface. The processing device may receive the input of the user and obtain the scanning region on the combination model. In some embodiments, scanning parameters of different portions of the target subject or different target subjects may be generated in advance and stored in the storage device, and the processing device may automatically obtain the scanning parameters based on user instructions determined by the combination model and a preset relationship between one or more characteristics (e. g., the thickness, the size, the type, etc.) of the target subject and one of the scanning parameters. For example, the user may indicate that the target subject needs to scan the lower limbs by the combination model, and the processing device may automatically retrieve relevant the scanning parameters of the lower limbs. The preset relationship may include a correspondence between the thickness of the lower limb and the exposure parameter.

In some embodiments, different portions of the target subject may be different in characteristics (e.g., different thicknesses of the different portions of the lower limbs). The same exposure parameter used by the second imaging device for scanning different portions of the target subject may result in inconsistent grayscale, brightness, and brightness of multiple obtained images, which may affect the visual effect of the final image composed of the multiple obtained images. The processing device may determine thicknesses of different portions of the target subject (e.g., the lower limbs) based on the combination model. The processing device may determine the exposure parameter based on the preset relationship (e.g., a correspondence between the thickness of the lower limbs and the exposure parameter), and obtain images of different portions of the target subject based on different exposure parameters, thereby improving the consistency of brightness and darkness of the determined images.

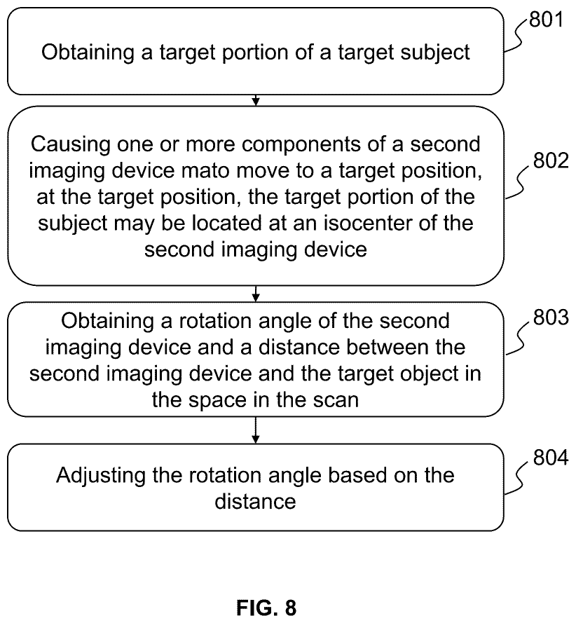

In some embodiments, the processing device may cause the second imaging device to perform the scan on the target subject based on the scanning parameters to obtain a target image. The scan manner may include a stepping acquisition process, a control process of the scan for a portion of a target subject, and other manners. More descriptions of the stepping acquisition manner may be found in FIG. 3-FIG. 7 and related descriptions. More descriptions of the control process of the scan for a portion of a target subject may be found in FIG. 8-FIG. 12 and related descriptions. The second imaging device may obtain at least two medical images of the target subject in the scan. For example, the second imaging device may rotate at least two of the components (e.g., the radiation source, the detectors, etc.) to obtain multiple groups of medical image data corresponding to different views of the target subject. As another example, a scanning table may be moved to obtain at least two medical image data corresponding to different scanning regions of the target subject.

In some embodiments, the processing device may cause the second imaging device to perform multiple rounds of scans on the target portion of the target subject based on the one or more scanning parameters. More descriptions of the second imaging device may be found in FIG. 1 and related descriptions.

The target portion of the target subject refers to a target portion of the target subject. The one or more scanning parameters corresponding to the multiple rounds of scans may be the same or different, and the processing device may be determined or obtained based on actual needs. The count of multiple rounds of scans may be set according to actual needs.

In some embodiments, the target portion may include the heart of the target subject. The causing a second imaging device to perform multiple rounds of scans on the target portion of the target subject based on the one or more scanning parameters may include causing the second imaging device to perform a rotation scan on the heart. The rotation scan may include the multiple rounds of scans, and each of the multiple rounds of scans may correspond to a rotation angle.

In some embodiments, the target portion may include a leg of the target subject, and the causing a second imaging device to perform multiple rounds of scans on the target portion of the target subject based on the one or more scanning parameters may include causing the second imaging device to perform a stepping scan on the leg. In some embodiments, The stepping scan may include multiple bed positions of scans, and each of the multiple rounds of scans may correspond to one bed position of scan in the multiple bed positions of scans.

In some embodiments of the present disclosure, by using the different scanning parameters in the multiple rounds of scans, the second imaging device may be controlled precisely to obtain multiple target images of the target portion of the target subject with more accuracy.

In some embodiments, the processing device may display the combination model on a user interface; obtain a first input of a user via the user interface generated according to the combination model; and determine the one or more scanning parameters based on the first input of the user.

The user interface may be configured to facilitate communication between the user and the processing device, the terminal device, the storage device, etc. For example, the user interface may display data (e.g., an analysis result, or an intermediate result) obtained and/or generated by the processing device. As a further example, the user interface may display the combination model. The user interface may be configured to receive user input from the users and/or the target subject. More descriptions of the user interface may be found in FIG. 1 and related descriptions.

The first input refers to instruction information related to the scanning parameters. In some embodiments, the first input may be in the form of a button input, a mouse input, a text input, a voice input, an image input, a touch screen input, a gesture command, an EEG, eye movement, or any other feasible instruction data.

In some embodiments, the first input may include a content related to the scanning region. For example, the first input may include a voice input instruction and/or an image input instruction for determining the scanning starting ending point position and the scanning ending point position. The image input instruction refers to an instruction that includes the scanning starting ending point position and the scanning ending point position through inputting an instruction image and for determining the scanning starting ending point position and the scanning ending point position. The instruction image corresponding to the image input instruction may be a portion of the combination model. For example, if the scanning region is the lower limbs, the instruction image corresponding to the image input instruction may be an image of the lower limbs in the combination model.

In some embodiments, the user may issue a voice instruction regarding the scanning starting ending point position, the scanning ending point position of the scanning region through voice input.

In some embodiments, the processing device may cause the terminal device (e.g., the user interface) to display the combination model. The users may draw the scanning region corresponding to the target portion of the target subject on the combination model of the user interface through the terminal device, and mark the scanning starting ending point position and scanning ending point position corresponding to the target portion of the target subject.

In some embodiments of the present disclosure, by obtaining the first input of the user based on the user interface displaying the combination model and determining the scanning parameters, the second imaging device may be controlled precisely to scan within the scanning region to obtain a target image of the target portion of the target subject, improving the acquisition efficiency and accuracy.

In some embodiments, the processing device may obtain a trained machine learning model; and determine the one or more scanning parameters of the target subject based on the combination model and the trained machine learning model.

The trained machine learning model refers to a scanning parameter model for determining the one or more scanning parameters of the target subject. In some embodiments, the trained machine learning model may be a neural network model. The selection of model types may depend on specific situations.

In some embodiments, an input of the trained machine learning model may include the combination model, and an output of the trained machine learning model may include the one or more scanning parameters of the target subject.

In some embodiments, the trained machine learning model may be trained based on multiple labeled training samples. For example, by inputting the multiple labeled training samples into an initial machine learning model, a loss function may be constructed based on the labels and output results of the initial machine learning model. Based on the loss function, parameters of the initial machine learning model may be iteratively updated through gradient descent or other manners until a termination condition is satisfied, and the trained machine learning model may be obtained. The termination condition may be a convergence of the loss function, the count of iterations reaching a threshold, or the like.

In some embodiments, the training samples may at least include a sample combination model corresponding to a sample target subject, and the training samples may be determined based on historical data. The labels of the training sample may be scanning parameters corresponding to the sample target subject. The labels may be obtained based on the processing device or manual annotation.

In some embodiments of the present disclosure, by using the trained machine learning model, the scanning parameters may be efficiently and accurately obtained, resulting in better results than manual settings. The trained machine learning model may be adapted to different types of target subjects, thereby further improving the efficiency of scanning.

In some embodiments, the processing device may obtain a second input of the user via the user interface generated according to the combination model, and adjust the one or more scanning parameters based on the second input of the user, more descriptions may be found in FIG. 3-FIG. 7.

In some embodiments, the one or more scanning parameters may include a scanning range defined by a starting point and/or an ending point, and the processing device may cause a second imaging device to arrive at the starting point and the ending point in a first image acquisition stage before a target portion of the target subject is injected with a contrast agent, and obtain a first image by causing the second imaging device to perform a scan. More descriptions regarding obtaining the first image may be found in FIG. 3-FIG. 7.

In some embodiments, the processing device may cause one or more components of a second imaging device to move to a target position. At the target position, the scanning range of the target subject may be located at an isocenter of the second imaging device. More descriptions regarding moving the one or more components of a second imaging device to a target position may be found in FIG. 8 and related descriptions.

In some embodiments, the one or more scanning parameters may include a rotation angle of one or more components of the second imaging device for performing a scan on the target subject, and the processing device may determine the rotation angle by adjusting an initial rotation angle. Mode descriptions for determining the rotation angle may be found in FIG. 8 and related descriptions.

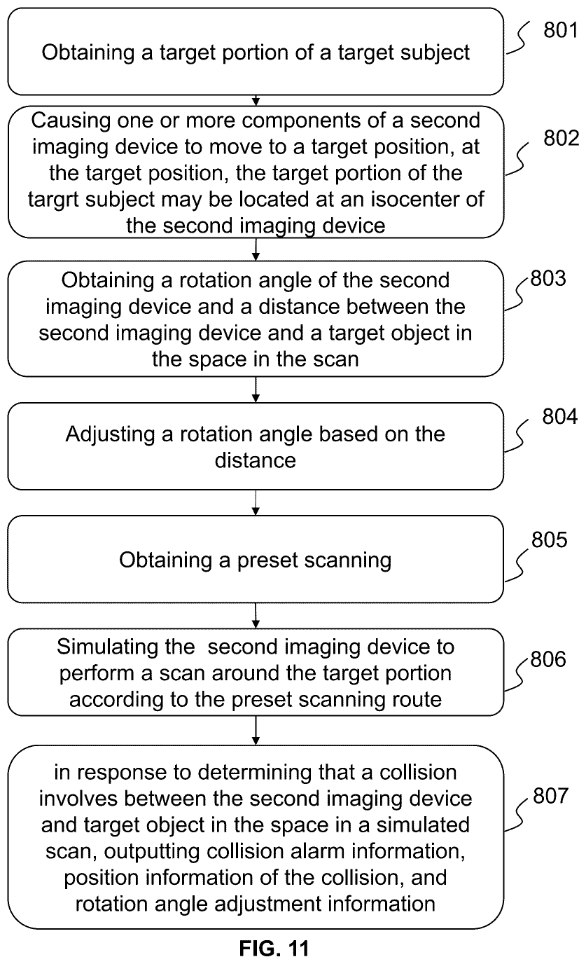

In some embodiments, the processing device may determine the scanning route by performing collision detection. The collision detection may include simulating a movement trajectory (i.e., a scanning route) of one or more components of the second imaging device, determining whether a collision involves when the one or more components of the second imaging device move along the simulated movement trajectory, and determining the scanning route based on the determination result. The collision may involve between an object in a movement trajectory of one or more components (e.g., a rack, a detector, or a scanning bed) of the second imaging device and the one or more components (e.g., a rack, a detector, or a scanning bed) of the second imaging device, or between a component and another component of the second imaging device, or between the target subject and one of the one or more component of the second imaging device, more descriptions may be found in FIG. 11 and related descriptions.

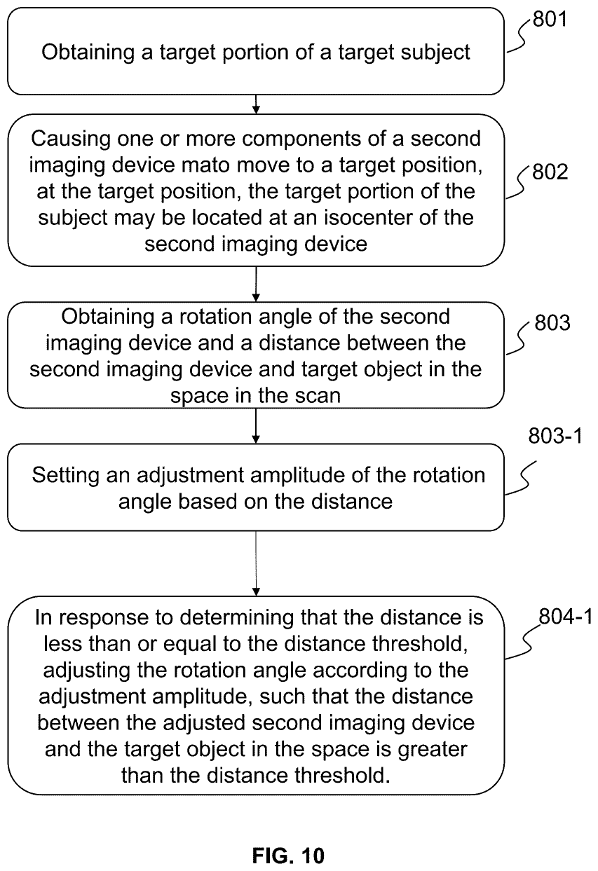

In some embodiments, the one or more scanning parameters may include a rotation angle for the one or more components of the second imaging device used to perform the scan on the target portion of the target subject, and the processing device may predict whether the collision involves in the scan based on the a distance between the target subject and the one or more components of the second imaging device; adjust the rotation angle in response to determining the collision involves in the scan; cause the second imaging device to perform the scan based on the adjusted rotation angle. More descriptions for determining the rotation angle may be found in FIG. 10 and related descriptions.

In some embodiments of the present disclosure, determining the scanning parameters based on the target portion of the target subject may be beneficial for controlling the second imaging device to obtain the target image of the target portion of the target subject accurately, reducing scanning acquisition time, radiation exposure time during the operation, and improving acquisition efficiency and accuracy. According to the 3D geometric model and anatomical structure model, the imaging device may position and adjust based on the real-time image without additional repeated operations for the user, which can reduce radiation exposure during the operation and improve functional execution efficiency.

It should be noted that the above description of the process is only for example and explanation, and does not limit the scope of application of this manual. For technical personnel in this field, various modifications and changes to the process can be made under the guidance of this manual. However, these modifications and changes are still within the scope of this manual.

FIG. 3 is a flowchart illustrating an exemplary process 300 for a stepping acquisition according to some embodiments of the present disclosure.

For example, for a digital subtraction angiography device, as shown in FIG. 3, the stepping acquisition process may include the following operations.

In 301, a target portion of the target subject may be obtained.

In some embodiments, by acquiring images (images of the external structure, the medical images, etc.) of the target subject (e.g., the patient) through the first imaging device and the second imaging device, the combination model of the target subject may be generated based on the images, and the target portion of the target subject may be obtained from the combination model of the target subject. More descriptions of the combination model and the target portion of the target subject may be found in FIG. 2 and related descriptions. More descriptions of the imaging device and the second imaging device may be found in FIG. 1 and related descriptions.

It should be noted that the combination model may be a 3D human body model, which may include a surface contour of a portion of the target subject and an internal anatomical structure of the target subject.

In some embodiments, the first imaging device may be a camera or other device for acquiring the images.

Additionally, for example, if the target portion of the target subject is double lower limbs, a detector in the second imaging device may select a horizontal detector, and if the target portion of the target subject is single lower limbs, a detector in the second imaging device may select a vertical detector.

In 302, a scanning starting ending point position and a scanning ending point position may be determined according to the target portion of the target subject.

In some embodiments, the scanning starting ending point position and scanning ending point position of the target portion of the target subject may be obtained based on a first input generated by a user via a user interface. For example, the scanning starting ending point position and scanning ending point position may be obtained based on a voice input instruction input by the user. In some embodiments, the voice input instruction may be input by the user interface, such as a voice input button.

As another example, the scanning starting ending point position and scanning ending point position may be obtained based on an image input instruction input by the user based on the target portion of the target subject represented in the combination model. The user may mark the target portion of the target subject on the combination thereof or mark the scanning starting ending point position and scanning ending point position on the combination model, and the marked combination model may serve as the first input. The processing device may obtain the first input and determine the scanning starting ending point position and scanning ending point position from the combination model directly.

In 303, a digital subtraction angiography device may be controlled to move to the scanning starting ending point position and/or scanning ending point position to obtain a target image of the target portion of the target subject.

In some embodiments, the processing device may associate a starting ending point position and an ending point position of the target portion of the target subject obtained by the imaging device and the second imaging device with the scanning starting ending point position and scanning ending point located by the digital subtraction angiography device.

In some embodiments, the processing device may obtain a second input of the user via the user interface generated according to the combination model; and adjust the one or more scanning parameters based on the second input of the user.

More descriptions of the combination model, the user interface, and the scanning parameters may be found in FIG. 2 and related descriptions.

The second input is an instruction for adjusting one of the scanning parameters. In some embodiments, the second input may include a voice input instruction and/or an image input instruction for adjusting one of the scanning parameters.

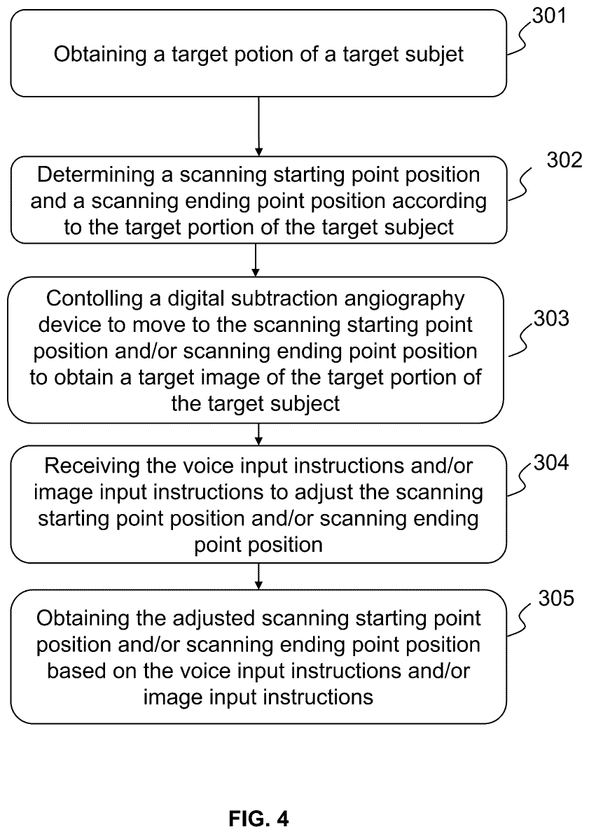

FIG. 4 is a flowchart illustrating an exemplary process 350 for a stepping acquisition according to some embodiments of the present disclosure. As shown in FIG. 4, in some embodiments, the stepping acquisition process may further include:

In 304, the voice input instruction and/or image input instruction may be received to adjust the scanning starting ending point position and/or scanning ending point position;

In 305, the adjusted scanning starting ending point position and/or scanning ending point position may be obtained based on the voice input instruction and/or image input instruction.

In some embodiments, the scanning starting ending point position and/or scanning ending point position may be adjusted through the voice input instruction and/or image input instruction to obtain a scanning region that meets the requirements.

In some embodiments of the present disclosure, by adjusting the one or more scanning parameters (e.g., the scanning starting ending point position and/or the adjusted scanning ending point position) through the second input (e.g., the voice input instructions and/or image input instructions), the accuracy of the determined scanning parameters can be improved and the accuracy of the subsequent target image obtained can be improved.

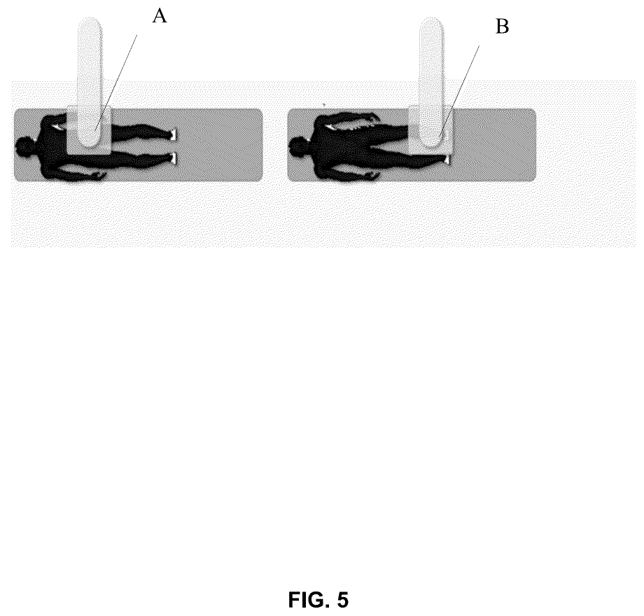

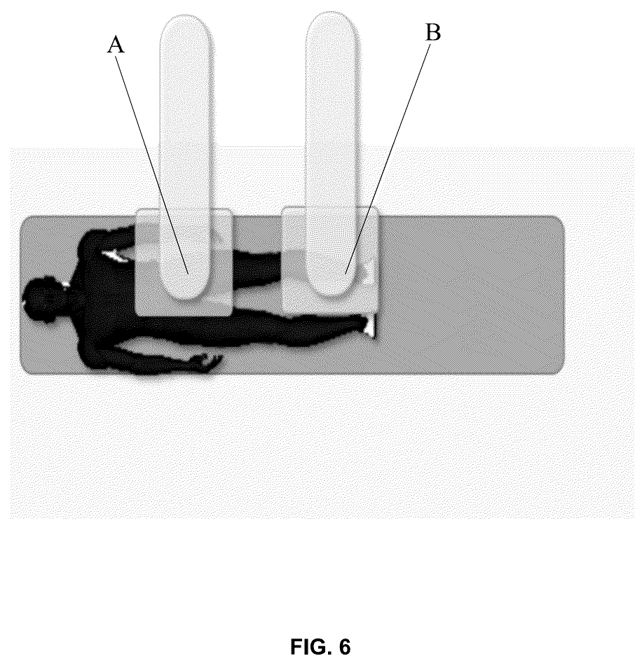

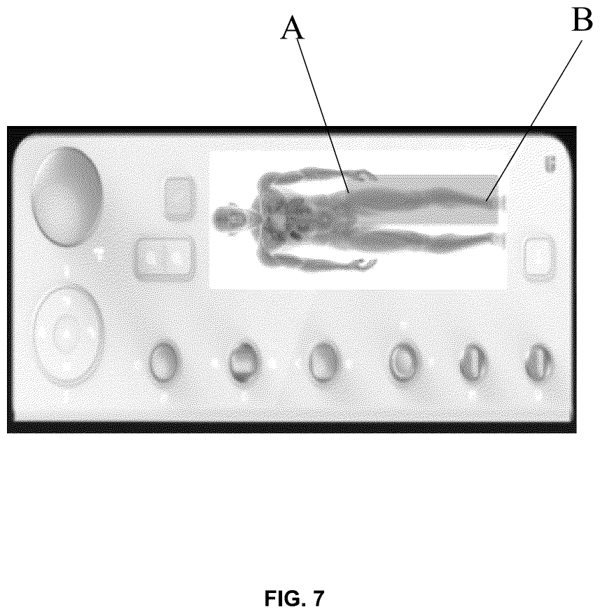

FIG. 5 is a schematic diagram illustrating an exemplary process for a stepping acquisition of a digital subtraction angiography (DSA) device according to some embodiments of the present disclosure. FIG. 6 is a schematic diagram illustrating another exemplary process for a stepping acquisition of a digital subtraction angiography (DSA) device according to some embodiments of the present disclosure. FIG. 7 is a schematic diagram illustrating another exemplary process for a stepping acquisition of a digital subtraction angiography (DSA) device according to some embodiments of the present disclosure.

For example, as shown in FIG. 5-FIG. 7, taking a left leg blood vessel with the target portion of the target subject as an example, the imaging device may automatically identify the target subject and display the combination model on the user interface (e.g., the user interface shown in FIG. 7). The anatomical structure model of the target subject (e.g., CTA (angiography) radiography or standard blood vessel model) may be overlaid on the 3D geometric model to obtain the combination model. When performing the stepping acquisition, for example, after the user (e.g., the doctor) selects a stepping acquisition protocol via, for example, the voice selection, the first input (e.g., a voice instruction, etc.) may be issued for determining the scanning starting ending point position and scanning ending point position, for example, as shown in FIG. 5-FIG. 7, the user may directly instruct “determining a position A of the left leg (e.g., the buttock) as a scanning staring position, and determining a position B (e.g., the ankle) as a scanning ending point position,” or may directly select the position A of the left leg (e.g., the buttock) as the scanning starting ending point position and the position B of the left leg (e.g., the ankle) as the scanning ending point position in the combination model in the user interface (UI) shown in FIG. 7. If the currently selected scanning starting point and scanning ending point position do not meet the actual needs, the user may adjust the current scanning starting ending point position and/or scanning ending point position through the second input (the voice input instructions and/or image input instructions) to obtain the adjusted scanning starting ending point position and/or scanning ending point position. After the scanning starting point and/or scanning ending point position are determined, the user may confirm the positions through the voice or click for confirming on the user interface. As shown in FIG. 5-FIG. 6, the processing device may designate the position A (e.g., the buttock) and the position B (e.g., the ankle) of the left leg as the scanning starting ending point position and scanning ending point position, respectively. For example, during a process of scanning the left leg and lower limbs, the processing device may control the digital subtraction angiography device to reach the position A, and adjust a distance between a C-arm upper detector and the lower limb of the left leg by raising and lowering the scanning bed or adjusting the distance between the C-arm upper detector and the left leg and lower limb to achieve an appropriate scanning position. The processing device may determine a scanning route based on the scanning region. The processing device may simulate whether a collision involves between the digital subtraction angiography device (e.g., the C-arm and/or rack) and surrounding objects or the target subject (e.g., the patient) according to a current scanning route. When determining that no collision involves in the scan, the processing device may automatically control the digital subtraction angiography device to perform the stepping acquisition based on the scanning region selected by the doctor. Specifically, when the scanning region matches a preset scanning region, in response to determining that the collision involves between the analog digital subtraction angiography device and a target object during a simulated stepping acquisition according to the scanning route, a reminder for the collision may be output. The processing device may adjust the position of the target object according to the reminder and/or the scanning route. In response to determining that no collision is involved between the analog-digital subtraction angiography device and the target object during the simulated stepping acquisition according to the scanning route, the processing device may trigger the exposure and control the digital subtraction angiography device to perform the stepping acquisition in the actual scanning region to obtain a target image of the target portion of the target subject. By performing the above process, the acquisition time of the stepping acquisition may be reduced, and the acquisition efficiency and accuracy can be improved.

In some embodiments, the one or more parameters may include a scanning range defined by a starting point and an ending point, the processing device may cause a second imaging device to arrive at the starting point and/or the ending point in a first image acquisition stage before a target portion of the target subject is injected with a contrast agent, and obtain a first image by causing the second imaging device to perform a scan.

More descriptions of the scanning region may be found in FIG. 2 and related descriptions.

More descriptions of the second imaging device may be found in FIG. 1 and related descriptions.

In some embodiments, the processing device may generate a control instruction based on the scanning starting ending point position and the scanning ending point position in the scanning region, and control the one or more other components (e.g., the detector) in the second imaging device to reach the scanning starting ending point position. By controlling the one or more other components of the second imaging device to adjust the positions of the one or more other components (e.g., lifting the scanning bed or adjusting the distance between the C-arm upper detector and the target subject), the target portion of the target subject (e.g., lower limbs, heart, etc.) may be reached at an appropriate scanning position. The processing device may further obtain scanning images of the target portion of the target subject by controlling the one or more other components of the second imaging device to move to the scanning ending point position, and determine a region from the scanning starting ending point position to the scanning ending point position as the scanning region of the target subject.

In some embodiments, the one or more components may include a C-arm, a scanning bed, a detector, a radiation source, or the like.

In some embodiments, the control instruction may include various parameters related to movement of the one or more other components in the second imaging device.

In some embodiments, the parameters related to movement may include a moving distance, a moving direction, a moving speed, or any combination thereof.

In some embodiments, the processing device may generate the control instruction based on the scanning starting ending point position and the scanning ending point position in the scanning region to control the second imaging device to perform the scan on the target subject.

The first image refers to a medical image before the target portion of the target subject is injected with the contrast agent.

In some embodiments, before the target portion of the target subject is injected with the contrast agent, the processing device may generate the control instructions based on the scanning starting point position and the scanning ending point position in the scanning region, control one or more other components in the second imaging device to move from the scanning starting point position to the scanning ending point position at a preset moving speed, and perform the scan to obtain a first image.

In some embodiments, the preset moving speed may be uniform, and the processing device may control the one or more other components of the second imaging device to start moving from the scanning starting point position. The plurality of first images may be obtained by acquiring images once a preset step size of movement reaches the scanning ending point position. The preset step size refers to a parameter value of the movement distance of the second imaging device.

In some embodiments in the present disclosure, before the target portion of the target subject is injected with the contrast agent, by controlling the movement of the second imaging device while performing the scan, the first image may be obtained, which can be used to determine the target image subsequently and improve the quality of the determined target image.

In some embodiments, operation 303 in the stepping acquisition process may further include:

In 303-0, in a first image acquisition stage before the target portion of the target subject is injected with the contrast agent, the digital subtraction angiography device may be controlled to move to the scanning starting ending point position and/or the scanning ending point position at a preset speed and acquire images to obtain a first image of the target portion of the target subject.

In some embodiments, the first image of the target portion of the target subject may be obtained by operating the digital subtraction angiography device at a preset speed and acquiring the images based on the first image acquisition stage before the target portion of the target subject is injected with the contrast agent. Specifically, in the first image acquisition stage before the target portion of the target subject is injected with the contrast agent (i.e., in a first mask sequence), in response to the user pressing an exposure button, the processing device may control the digital subtraction angiography device to move from the scanning starting ending point position to the scanning ending point position selected by the user at a preset speed and acquire the images. In addition, same step sizes may trigger the exposure acquisition during the movement.

It should be noted that the preset speed may be set to a constant speed.

In some embodiments, the processing device may determine a blood flow velocity of the target portion in a second image acquisition stage after the target portion of the target subject is injected with the contrast agent; adjust a moving speed of the second imaging device based on the blood flow velocity; and obtain a second image by causing the second imaging device to perform a scan.

The target portion refers to a target imaging portion of the target subject.

More descriptions of the target portion may be found in FIG. 2 and related descriptions.

More descriptions of the second imaging device and the target subject may be found in FIG. 1 and related descriptions.

The blood flow velocity refers to the blood volume flowing through a vascular cross-section of the target portion per unit time. For example, the blood flow velocity is A milliliters per second, and A is a value. As another example, the blood flow velocity may include an average blood flow velocity of all blood vessels within the target portion.

The blood flow velocity may be determined in multiple ways. In some embodiments, the processing device may obtain a historical blood flow velocity of a historical target portion that is the same as the blood flow velocity of the target portion and stored in the storage device, and determine the historical blood flow velocity as a current blood flow velocity of the target portion.

The second image refers to a medical image after the target portion of the target subject is injected with the contrast agent.

In some embodiments, the processing device may determine a time point when the contrast agent reaches the next exposure position based on the position of the contrast agent in the second image and the blood flow velocity of the target portion; based on the time point, a control instruction may be generated to adjust a motion speed of each of the one or more other components (e.g., the radiation source, the detector, the C arm, the gantry, etc.) of the second imaging device, so that the one or more other components of the second imaging device may move to the next exposure position at or before the time point.

In some embodiments, in addition to adjusting the motion speed of the second imaging device, the processing device may adjust the motion speed of the scanning bed. The processing device may adjust the motion speed of the scanning bed based on the blood flow velocity of the target portion so that the target portion after injecting with the contrast agent is located in the imaging region of the second imaging device.

The exposure position may be a position that the second imaging device reaches the scanning region. The exposure position may be determined according to the actual situation.

In some embodiments of the present disclosure, by adjusting the motion speed of each of the one or more other components of the second imaging device based on the blood flow velocity, the second image obtained through the radiography may be clearer and more accurate, which can avoid affecting subsequent processing of the second image.

In some embodiments, the operation 303 in the stepping acquisition process may further include the following operations.

In 303-1, a blood flow velocity of the target portion may be obtained in a second image acquisition stage after the target portion of the target subject is injected with the contrast agent;

In 303-2, a second image of the target portion may be obtained by controlling the digital subtraction angiography device to move to the scanning starting ending point position and/or the scanning ending point position and acquiring one or more images according to the blood flow velocity of the target portion.

In some embodiments, the processing device may obtain the second image of the target portion by controlling the digital subtraction angiography device to move within the scanning region and acquiring the images according to the obtained blood flow velocity merely in the second image acquisition stage after the target portion of the target subject is injected with the contrast agent. For example, taking the lower limb blood vessels of the target subject as the target portion, in the second image acquisition stage after the target portion of the target subject is injected with the contrast agent (i.e., in a second phase of contrast agent acquisition sequence), the user (e.g., the doctor) may manually or automatically trigger a motion mode to control the digital subtraction angiography device to move within the scanning region and acquire the images. In the motion mode triggered manually, the user may determine an arrival position of the contrast agent in the lower limb blood vessels based on the acquired images, and control the motion speed of the digital subtraction angiography device by manually pressing a movement control button; in the motion mode triggered automatically, in response to the user presses the movement control button, the processing device may determine the arrival position of the contrast agent in the lower limb blood vessels through the acquired images, determine a time point when the contrast agent reaches the next exposure position based on the blood flow velocity of the lower limb, and automatically control the motion speed of the digital subtraction angiography device.

In some embodiments, the processing device may obtain a third image of the target portion of the target subject; and determine the blood flow velocity based on the third image.

The third images may include images of the contrast agent at the different positions on the target portion. The third images may include multiple images, the multiple images may have a chronological order, which reflects different positions of contrast agents at the target portion of the target subject.

In some embodiments, the processing device may utilize a digital tracking technique to measure a moving distance and/or a moving duration of a marked point in a third image of two frames before and after or the third image with an interval of N frames in the target portion, and automatically determine the blood flow velocity based on the moving distance and/or the moving duration.

The marked point refers to a position previously selected in the third image. For example, the marked point may be a pixel with the highest grayscale value, a pixel with a median grayscale value, etc., or a feature point. In some embodiments, the larger the grayscale value in the third image, the higher the quality of the contrast agent at the position may be.

In some embodiments of the present disclosure, by determining the blood flow velocity, a reliable and efficient calculation basis may be provided for a motion velocity of each of the one or more other components of the second imaging device.

In some embodiments, the operation 303-1 of the stepping acquisition process may further include following operations.

In 303-1-1, a third image of the target portion may be obtained in the second image acquisition stage after the target portion of the target subject is injected with the contrast agent;

In 303-1-2, grayscale values of the third images, when the contrast agent is at different positions, may be obtained based on the third images;

In 303-1-3, the blood flow velocity of the target portion may be determined based on the grayscale values of the third images of the contrast agent at different positions.

In some embodiments, the processing device may determine a moving distance and a moving duration corresponding to the moving distance of the contrast agent in the target subject; and determine the blood flow velocity based on the moving distance and the moving duration corresponding to the moving distance of the contrast agent in the target subject.

The moving distance refers to a distance that the contrast agent moves in the target portion.

The moving duration refers to a duration required for the contrast agent to complete the moving distance in the target portion.

In some embodiments, the processing device may determine a pixel distance of the marked point moving in two frames of the third image with a preset count of frames, and determine an actual distance corresponding to the pixel distance as the moving distance of the contrast agent moving in the target portion. The moving duration may be obtained based on the preset number of frames and the frame rate of the third image. The blood flow velocity may be determined based on a ratio of the moving distance and the moving duration corresponding to the moving distance. The preset number of frames may be a system preset value, a manual preset value, or the like.

In some embodiments of the present disclosure, by determining the blood flow velocity through the moving distance and the moving duration corresponding to the moving distance, the efficiency of calculation can be improved while a relatively high accurate blood flow velocity may be obtained.

In some embodiments, the operation 303-1 in the stepping acquisition process may further include following operations.

In 3Mar. 1, 2011, a moving distance of the contrast agent at the target portion and a moving duration corresponding to the moving distance may be obtained in the second image acquisition stage after the target portion of the target subject is injected with the contrast agent;

In 3Mar. 1, 2012, the blood flow velocity of the target portion may be determined based on the moving distance and the moving duration.

The embodiment may determine the scanning starting ending point position and the scanning ending point position based on the target portion, and the digital subtraction angiography device may be moved to the scanning starting ending point position and/or scanning ending point position to obtain a target image of the target portion, which can reduce an acquisition time of the stepping acquisition, and improve acquisition efficiency and accuracy.

In some embodiments, the processing device may obtain a target image of the target portion of the target subject based on the first image and the second image.

The target image refers to a final medical image of the target portion.

In some embodiments, the processing device may generate a plurality of medical images at different stages of performing the scan on the target subject. Merely for example, the scan may be a DSA scan configured to imaging the blood vessels of the lower limbs of the target subject. For the DSA scan, the contrast agent may be injected into the target subject. The plurality of medical images may include a first image obtained before the contrast agent is injected into the target subject and a second image obtained after the contrast agent is injected into the target subject. Merely for example, assuming that the second image is obtained at a second time point after the first image is obtained at a first time point. In some embodiments, the first image may be used as a mask, and if the target subject remains stationary during a time period between the first time point and the second time point, a difference image between the second image and the first image may show the blood vessels of the target subject without showing other organs or tissues.

It should be noted that if the target subject moves between the first time point and the second time point, the quality of the final obtained difference image (e.g., causing motion artifacts in the image) may be affected.

In some embodiments of the present disclosure, the target image may be obtained based on the first image and the second image, and an imaging effect of the internal structure may be enlarged and clearly displayed, which is conducive to the analysis and diagnosis of diseases (e.g., vascular diseases, tumors, etc.).

In some embodiments, operation 303 in the stepping acquisition operation may further include the following operations.

In 303-0, in the first image acquisition stage before the target portion of the target subject is injected with the contrast agent, a first image of the target portion of the target subject may be obtained by controlling the digital subtraction angiography device to move to the scanning starting ending point position and/or the scanning ending point position at a preset speed.

In 303-1, in the second image acquisition stage after the target portion of the target subject is injected with the contrast agent, a blood flow velocity of the target portion may be obtained.

In 303-2, a second image of the target portion of the target subject may be obtained by controlling the digital subtraction angiography device to move to the scanning starting ending point position and/or the scanning ending point position according to the blood flow velocity of the target portion.

In 303-3, a target image of the target portion may be obtained according to the first image and the second image.

In some embodiments, the processing device may obtain the target image of the target portion by operating the digital subtraction angiography device and acquiring the images simultaneously in the first image acquisition stage before the target portion of the target subject is injected with the contrast agent and in the second image acquisition stage after the target portion of the target subject is injected with the contrast agent. Specifically, for the first image acquisition stage before the target portion of the target subject is injected with the contrast agent (i.e., the first mask sequence) and the second image acquisition stage after the target portion of the target subject is injected with the contrast agent (i.e., the second contrast agent acquisition sequence), in the first mask sequence, the processing device may control the digital subtraction angiography device to move to the scanning starting ending point position and/or the scanning ending point position and acquiring the images. After the first mask sequence, the processing device may control the digital subtraction angiography device to perform the second contrast agent acquisition sequence, at this time, a scanning starting ending point position of the second contrast agent acquisition sequence may be a scanning ending point position of the first mask sequence, and a scanning ending point position of the second contrast agent acquisition sequence may be a scanning starting ending point position of the first mask sequence, i.e., the digital subtraction angiography device may operate in reverse during a second contrast agent acquisition sequence and acquire images, in addition, a path and position of the digital subtraction angiography device during the second path and location of the digital subtraction angiography device during the second contrast agent acquisition sequence may be the same as a path and position of the digital subtraction angiography device during the first mask sequence.

It should be noted that in the first mask sequence, the digital subtraction angiography device may operate at a preset speed (e.g., a constant speed), and in the second contrast agent acquisition sequence, the digital subtraction angiography device may operate at a variable speed (e.g., according to the blood flow velocity, the digital subtraction angiography device may be controlled to move within the target scanning region and acquire the images).

FIG. 8 is a flowchart 1 illustrating an exemplary process for controlling a scan of a portion of a target subject according to some embodiments of the present disclosure.

For example, the processing device may perform the process for controlling the scan of a portion of the target subject based on the following operations, as shown in FIG. 8, the controlling process may include:

In 801, a target portion of a target subject may be obtained;

In some embodiments, the target portion may be the heart of the target subject or other portions of the target subject, which may not be limited herein. More descriptions of obtaining the target portion of the target subject may be found in FIG. 3 and related descriptions.

In 802, one or more components of a second imaging device may be caused to move to a target position. At the target position, the target portion of the target subject may be located at an isocenter of the second imaging device;

In some embodiments, the C-arm may be caused to move to the target position according to a received control instruction.

In some embodiments, the one or more scanning parameters may include a scanning range. The processing device may cause one or more components of a second imaging device to move to the target position. At the target position, the scanning range of the target subject may be located at an isocenter of the second imaging device. The processing device may cause the second imaging device to perform a scan on the target subject.

More description of the target subject and the second imaging device may be found in FIG. 1 and related descriptions.

More descriptions of the scanning region may be found in FIG. 2 and related descriptions.

The target position reflects a corresponding relationship between the scanning region of the target subject and the isocenter of the second imaging device. In some embodiments, when the target subject is located at the target position, the scanning region of the target subject may be located at the isocenter of the second imaging device. In some embodiments, when the target subject is located at the target portion, a geometric center of the scanning region may coincide (approximately) with the isocenter of the second imaging device, i.e., a deviation between the geometric center of the scanning region and the isocenter of the scanning region of the second imaging device may be less than a threshold (e.g., 10%, 5%, or 5 mm, 3 mm, 1 mm, etc.).

The isocenter refers to an imaging isocenter point of the second imaging device. The imaging isocenter point refers to the center of the imaging region.

In some embodiments, the second imaging device may have an isocenter. The isocenter point of the second imaging device refers to the mechanical isocenter of the second imaging device. For example, for the X-ray imaging device with the rack (e.g., a cylindrical rack, a C-arm), the imaging isocenter of the X-ray imaging device may be the center of the rack.

In some embodiments, the processing device may determine the scanning region of the target subject and determine a geometric center of the scanning region. The processing device may determine the target portion based on the geometric center and the isocenter of the second imaging device. For example, the processing device may obtain the scanning region of the target subject selected by medical staff through the user interface of the terminal device, determine the geometric center of the scanning region, and determine a position where the geometric center coincides with the isocenter as a target portion by adjusting the position of the target subject or respective positions of one or more components of the second imaging device.

In some embodiments, the processing device may generate a control instruction to control the second imaging device to perform the scan on the target subject when determining that the scanning region of the target subject is located at the isocenter of the second imaging device. For example, if the control instruction is to perform an isocenter rotation, the one or more components of the second imaging device (e.g., a radiation source, a rack) may rotate around the isocenter to perform a scan on the target subject; if the control instruction is to perform a non-isocenter point rotation, and the one or more components of the second imaging device may rotate around points other than isocenter point to perform a scan on the target subject.

In some embodiments of the present disclosure, by determining a position of the isocenter, there is no need for on-site operation and adjustment by the users, which can reduce the workload and waste of manpower and material resources.

In 803, a rotation angle of the second imaging device and a distance between the second imaging device and a target object in the space (e.g., a treatment room, a scanning room, etc.) may be obtained in the scan.

In some embodiments, the target subject in the space (e.g., a treatment room, a scanning room, etc.) may be a target object or a medical device, or a portion thereof.

In some embodiments, the medical device may include at least one of a scanning bed, a surgical display, a high-pressure injector, an electrocardiogram monitoring, a surgical cart, etc.

In some embodiments, all rotation angles of the C-arm and a distance between the C-arm and the target subject or medical device in the scan may be obtained.

For example, the distance between the C-arm and the target subject may be obtained. Specifically, a distance between the lowest end of the detector on the C-arm and the highest position on the surface of the target subject may be the distance between the C-arm and the target subject.

It should be noted that the processing device in some embodiments can control at least one movement in the C-arm and/or the scanning bed to locate the target portion at the isocenter of the second imaging device.

In some embodiments, the one or more scanning parameters may include a rotation angle of one or more components of the second imaging device for performing the scan on the target subject. The processing device may determine the rotation angle by adjusting an initial rotation angle.

In some embodiments, the one or more components may include a rack, radiation source, a detector of the second imaging device, or the like.

More descriptions of the target subject and the second imaging device may be found in FIG. 1 and related descriptions.

More descriptions of the combination model and the scanning parameters may be found in FIG. 2 and related descriptions.

The initial rotation angle may be a preset rotation angle, or a rotation angle in a current scan (e.g., a rotation angle at the current time, etc.).

The rotation angle may be configured to characterize an angle at which the radiation source and the detector rotate around a rotation center of the second imaging device. The rotation center may be at the isocenter of the second imaging device or a position other than the isocenter of the second imaging device.

In some embodiments, the initial rotation angle may include angle information of the rotation angles of each scanning position. For example, the initial rotation angle may be represented by vectors as (A1, A2, . . . ), wherein the vector element A1 represents information about the rotation angle at scanning position A1, and the vector element A2 represents information about the rotation angle at scanning position A2.

In some embodiments, when the rack of the second imaging device rotates to change the rotation angle, the scanning angle of the second imaging device may also change, resulting in a change in the position of the target subject relative to the detector, thereby obtaining the medical images of the target subject corresponding to different scanning angles. As another example, the user may manually adjust the positions of the radiation source and/or detector of the second imaging device to change the scanning angle.

The processing device may adjust the initial rotation angle in various ways to determine the rotation angle. In some embodiments, the adjustment process may include an amplitude adjustment, a direction adjustment, or the like. The amplitude adjustment refers to adjusting the magnitude of the rotation angle. The direction adjustment may be configured to determine a direction of adjustment, such as increasing or decreasing the initial rotation angle.

In some embodiments, the direction adjustment may be determined based on a preset rule. Merely for example, the preset rule may include: after rotating according to the adjusted rotation angle, increasing the distance between the target subject and the one or more components of the second imaging device. The adjustment amplitude may be determined based on a preset correspondence between different reference distances and different reference rotation angles.

In some embodiments, the processing device may also adjust the initial rotation angle based on a weight and a weight threshold of the target subject. For example, when the weight of the target subject is greater than the weight threshold, the initial rotation angle may be reduced. When the weight of the target subject is less than the weight threshold, the initial rotation angle may be increased. The weight threshold may be a system default value, a manual preset value, or the like.

In some embodiments, the processing device may also adjust only the angle information of a certain scanning position according to the actual situation.

In some embodiments of the present disclosure, the second imaging device may move within the scanning region (e.g., a linear motion or a rotational motion) to complete the scan. Therefore, the initial rotation angle may be adjusted by combining the model, which can avoid a collision between the components of the second imaging device and the target object in the environment where the target subject is located, distract attention of the user, ensure smooth progress of the scan, and improve the efficiency and safety of the scan.

In 804, the rotation angle may be adjusted based on the distance.

In some embodiments, a distance threshold may be set according to the actual situation, which may not be limited herein.

In some embodiments, after the target portion is located at the isocenter of the second imaging device, the rotation angle may be adjusted based on the distance between the obtained second imaging device and the target object in the space (e.g., a treatment room, a scanning room, etc.), which can correct the rotation angle more accurately.

In some embodiments, a rotation angle of the C-arm may be adjusted through the distance between the C-arm and the target subject or medical device, which not only can avoid the collision between the C-arm and the target subject or medical device, but also achieve the imaging better (i.e., by scanning the target portion more comprehensively with a relatively large rotation angle).

In some embodiments, the processing device may determine a distance between the target subject and the one or more components of the second imaging device based on the combination model; and adjust the initial rotation angle based on the distance.

In some embodiments, the distance between the target subject and a component of the second imaging device may be determined based on position information of the target subject and position information of the component of the second imaging device in a same coordinate system (also referred to as a reference coordinate system), e.g., a coordinate system applied to the second imaging device. The position information of the target subject in the reference coordinate system may be determined based on the combination model. As used herein, determining the position information of the target subject in the reference coordinate system may include determining the position information of the target subject in the reference coordinate system based on at least a portion of the combination model, such as the 3D geometric model, the anatomical model, or the combination model. The combination model, the 3D geometric model, and the anatomical model may indicate a contour of the target subject and the position information of points on the contour of the target subject in a coordinate system (also referred to as a first coordinate system) applied to the combination model, or in a coordinate system (also referred to as a second coordinate system) applied to the geometric model, or in a coordinate system (also referred to as a third coordinate system) applied to the anatomical model. The processing device may determine the position information of the target subject in the reference coordinate system based on a transform relationship between the first coordinate system and the reference coordinate system. For example, the processing device may transform the position information of the target subject in the second coordinate system applied to the 3D geometric model to spatial position information in the reference coordinate system of the second image device based on a transform relationship between the second coordinate system applied to the 3D geometric model and the reference coordinate system of the second image device. As another example, the processing device may transform position information of the target subject in the combination model to the spatial position information in the reference coordinate system of the second image device based on a transform relationship between the third coordinate system of the anatomical model and the reference coordinate system of the second image device.

In some embodiments, the processing device may also obtain a three-dimensional (3D) spatial model, and determine a distance between the target subject and the one or more components of the second imaging device based on the combination model and the 3D spatial model.

The 3D spatial model refers to a 3D model that represents an internal scenario of the environment where the target subject is located. In some embodiments, the 3D spatial model of the environment where the target subject is located may be used to represent an internal spatial structure and a target object located within the environment. The target subject may include medical devices already placed in the environment and/or medical devices or living organisms to be placed in the environment. Merely for example, the target object may include the first imaging device, the second imaging devices, target subjects, doctors, surgical displays, high-pressure syringes, electrocardiogram monitoring, surgical carts, or the like. In the 3D spatial model, a size of the target object may be proportionally reduced.

In some embodiments, the 3D spatial model of the environment where the target subject is located may be generated based on multiple 2D images. In some embodiments, the multiple 2D images may be images captured in advance through a camera. The processing device may obtain the 3D spatial model through 3D reconstruction technique based on the multiple pre-captured 2D images. The exemplary 3D reconstruction technique may include a texture shape (SFT) process, a shading reconstruction 3D shape process, a multi view stereo (MVS) process, a motion recovery structure (SFM) process, a time of flight (ToF) process, a structured light process, a Moir e schlieren process, or any combination thereof.

In some embodiments, the processing device may use other processes to obtain a 3D spatial model. For example, a depth sensor may be configured to obtain the depth data of the scenario in the environment where the target subject is located. The processing device may obtain the 3D spatial model based on the depth data of the environment where the target subject is located. For example, manual drawing, surveying, and other processes may be configured to obtain the 3D spatial model, which may not be limited herein.

In some embodiments, the processing device may generate the 3D geometric model of the target object based on data related to the target object (e.g., images of the target subject, medical images). The processing device may also fuse the 3D model of the target subject with the 3D spatial model of the environment where the target subject is located based on an intended position of the target subject, or integrate the combination model of the target subject with the 3D spatial model. The fused 3D spatial model may not only represent an appearance (e.g., pose, shape, size) and the internal structure of the target subject but also represent environmental data where the target subject is located. The intended position of the target subject refers to a position of the target subject to be reached in the environment.

In some embodiments, the 3D spatial model may be generated before or in the scan on the target subject. Optionally, image data of the target subject and environment where the target subject is located may be acquired in real-time, continuously, or intermittently (e.g., periodically), and the 3D spatial model may be updated continuously or intermittently (e.g., periodically) based on the image data.

The distance refers to one or more distance between one or more target objects (e.g., the target subject) and one or more components of the second imaging device. In some embodiments, the distance may be represented by a vector ((b1, y1), (b2, y2), . . . ), which represents a distance between a target object b1 and a component y1, a distance between a target object b2 and a component y2, or the like.

More descriptions of other objects may be found in FIG. 2 and related descriptions.

In some embodiments, the processing device may determine the distance between other objects (e.g., the target subject) and one or more components of the second imaging device based on the 3D spatial model. For example, the processing device may identify a first pixel representing one or more components of the second imaging device from the 3D spatial model, and may also identify a second pixel representing other objects from the 3D spatial model. The processing device may determine a pixel distance between the first pixel and the second pixel, and designate the pixel distance or the actual distance corresponding to the pixel distance as the distance.

In some embodiments, the processing device may also determine the distance based on a distance sensor before or in the scan and/or treatment of the target subject. The distance sensor may be installed on at least one target object within the environment where the target subject is located, such as a radiation source, a detector, a treatment head, an electronic portal imaging device (EPID), a workbench, ground, ceiling, or the like. The distance sensor may detect a distance between different target objects, such as detecting a distance between components of the second imaging device and the target subject, workbench, ground, ceiling, or the like. The distance sensor may include a capacitive distance sensor, an eddy current distance sensor, a Hall effect distance sensor, a Doppler effect distance sensor, or the like.

In some embodiments, the processing device may obtain a corresponding relationship (e.g., a table lookup) between different reference distances and different reference rotation angles, determine the rotation angle based on the distance through the corresponding relationship, and update the initial rotation angle.

In some embodiments of the present disclosure, based on the combination model, whether a collision involves between the target subject and one or more components of the second imaging device may be predicted. By adjusting the rotation angle, the probability of the collision can be reduced, and the efficiency of acquisition can be improved.

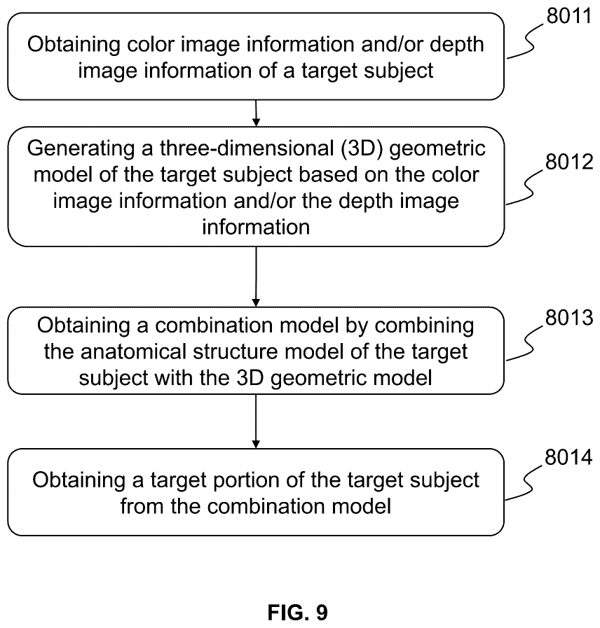

FIG. 9 is a flowchart illustrating an exemplary process for controlling a scan of a portion of a target subject according to some embodiments of the present disclosure. Operation 801 may be implemented according to process 900.

In 8011, color image information and/or depth image information of the target subject may be obtained.

In some embodiments, the color image information and/or the depth image information of the target subject may be obtained through an imaging device.

It should be noted that imaging devices may be a camera or other devices capable of obtaining image information. In addition, the camera may include a RGB color camera and a depth camera.

In 8012, a 3D geometric model of the target subject may be generated based on the color image information and/or the depth image information.

In some embodiments, the 3D geometric model may be a 3D human body model.

In 8013, a combination model may be obtained by combining an anatomical structure model of the target subject with the 3D geometric model.

In some embodiments, the anatomical structure model may be obtained through a CT scanner, a cone beam CT (CBCT) scanner, or a camera, i.e., the internal organ structure information of the target portion of the target subject may be obtained through CT or CBCT scan, and body surface information of a portion of the target subject may be obtained and obtained through the camera.

In 8014, a target portion of the target subject may be obtained from the combination model.

In some embodiments, the surface of a portion of the target subject may be seen from the combination model while the internal organ structure of the target subject may also be seen.

In some embodiments, by obtaining information about the target subject and the spatial environment of the space (e.g., a treatment room, a scanning room, etc.) through the camera, the combination model, the posture, and the 3D spatial model of the space (e.g., a treatment room, a scanning room, etc.) (e.g., an operating room) may be generated and identified. The combination model, the posture, and the 3D spatial model may be used to achieve functions of automatic heart rotation acquisition and control, i.e., the internal organ structure information of a portion of the target subject may be obtained through the CT or CBCT scan. The body surface information of the target portion of the target subject may be acquired and obtained through the camera, and the combination model may be generated. The spatial environment of the space (e.g., a treatment room, a scanning room, etc.) may be identified and whether a collision involves between the one or more components of the second imaging device (e.g., the C-arm) or between a target subject (e.g., a patient, a doctor, an operator, etc.) and the one or more components of the second imaging device in the space (e.g., a treatment room, a scanning room, etc.) (e.g., a scanning bed, a surgical display, a high-pressure injector, an electrocardiogram monitoring, a surgical cart, etc.) may be monitored in real-time simultaneously.

In some embodiments, a plurality of cameras may be arranged in the space (e.g., a treatment room, a scanning room, etc.). For example, taking arranging three cameras as an example, the three cameras may obtain body surface information of the target portion of the target subject and environmental information of the space (e.g., a treatment room, a scanning room, etc.) simultaneously. A camera that meets the clarity requirements from the three cameras may be selected, and the body surface information of the target portion and the environmental information of the space (e.g., a treatment room, a scanning room, etc.) may be acquired through the camera that meets the clarity requirements, the remaining two cameras may be used as a backup.

In some embodiments, the operation 804 in the process of controlling the scan of a portion of the target subject may include in response to determining that the distance is less than or equal to the distance threshold, adjusting the rotation angle such that the distance between the adjusted second imaging device and the target object in the space (e.g., a treatment room, a scanning room, etc.) is greater than the distance threshold.

FIG. 10 is a flowchart illustrating an exemplary process for controlling a scan of a portion of a target subject according to some embodiments of the present disclosure.

In some embodiments, as shown in FIG. 10, after the operation 803, the process for controlling the scan of a portion of the target subject may include following operations.

In 803-1, an adjustment amplitude of the rotation angle may be set based on the distance.

The operation 804 may further include 804-1, in response to determining that the distance is less than or equal to the distance threshold, the rotation angle may be adjusted according to the adjustment amplitude, such that the distance between the adjusted second imaging device and the target object in the space (e.g., a treatment room, a scanning room, etc.) is greater than the distance threshold.

In some embodiments, the greater a difference between the distance and the distance threshold, the greater the adjustment amplitude of the rotation angle may be.

In some embodiments, in response to determining that the distance is less than a distance threshold, the processing device may adjust the initial rotation angle to obtain the rotation angle, a distance between the target subject and the one or more components of the second imaging device after the initial rotation angle may be adjusted exceeding the distance threshold.

The distance threshold is a threshold condition related to the distance. In some embodiments, the distance threshold may be a system default value or a manually preset value. In some embodiments, the distance threshold may be determined based on the actual situations.

In some embodiments, the processing device may determine a relationship between the distance and the distance threshold in real-time before and/or in the scan. In response to determining that the distance is less than the distance threshold, the processing device may determine that a collision is involved in the scan. In response to determining that the distance is greater than the distance threshold, the processing device may determine that the collision dis involved in the scan.

In some embodiments, the processing device may use various processes such as manual analysis, theoretical calculation, and/or modeling to determine an adjustment amplitude and an adjustment direction in response to determining that the distance being is less than the distance threshold, the initial rotation angle may be adjusted based on the adjustment amplitude and the adjustment direction to obtain a rotation angle, so that after one or more components of the second imaging device rotate based on the rotation angle, a distance between the target subject and the one or more components of the second imaging device may exceed the distance threshold.

In some embodiments of the present disclosure, by determining that the distance is less than the distance threshold, the rotation angle may be adjusted in a timely manner to avoid the collision in the scan and improve the efficiency and safety of the imaging.

FIG. 11 is a flowchart 3 illustrating an exemplary process for controlling a scan of a portion of a target subject according to some embodiments of the present disclosure.

In some embodiments, as shown in FIG. 11, the process for controlling the scan of a portion of the target subject after the operation 804 may further include following operations.

In 805, a preset scanning route may be obtained;

In 806, the second imaging device may be simulated to perform a scan around the target portion according to the preset scanning route;

In 807, in response to determining that a collision involves between the second imaging device and the target object in the space (e.g., a treatment room, a scanning room, etc.) in a simulated scan, collision alarm information, position information of the collision, and/or rotation angle adjustment information may be output.

In some embodiments, a preset scanning route may be obtained by the processing device and a scan may be performed around the target portion by simulating the second imaging device according to the preset scanning route. In response to determining that a collision involves between the second imaging device and the target object in the space (e.g., a treatment room, a scanning room, etc.) in the simulated scan, the collision alarm information, the position information of the collision, and the rotation angle adjustment information may be output to adjust the rotation angle in real-time according to the actual situation, to avoid the collision between the second imaging device and the target object t in the space (e.g., a treatment room, a scanning room, etc.).

In some embodiments, the one or more scanning parameters may include a scanning route of one or more components of the second imaging device for performing a scan on the target subject. The scanning route may indicate a moving trajectory of the one or more components of the second imaging device. The processing device may predict whether a collision involves in the scan based on the scanning route. In response to determining that the collision involves in the scan, the processing device may adjust the scanning route and cause the second imaging device to perform the scan based on the adjusted scanning route.

More descriptions of the target subject and the second imaging device may be found in FIG. 1 and related descriptions.

The scanning route refers to the movement trajectory of the one or more components of the second imaging device in the scan on the target subject. For example, the scanning route may include parameters such as a starting point position of the component, an ending point position of the component, a moving speed, a moving direction of each component at least two time points in the scan, and a moving distance of the component within a time interval.

In some embodiments, the scanning route of the component may be stored in the storage device. The processing device may obtain the scanning route of the component from the storage device. In some embodiments, the processing device may determine the scanning route based on an imaging protocol of the target subject.

The imaging protocol refers to information related to scanning parameters associated with the scan and/or image reconstruction parameters associated with the scan. The imaging protocol may include scanning parameters. The scanning parameters may be various parameters used by the second imaging device for the scan. The second imaging device may set and adjust various components based on the scanning parameters. In some embodiments, the second imaging device may include multiple imaging protocols. Different target portions may correspond to different imaging protocols. The same target portion may include different imaging protocols. Merely for example, the imaging protocol may include a spinal axial scanning imaging protocol, an abdominal spiral imaging protocol, a cardiac rotation scanning protocol, or the like. In some embodiments, after determining the imaging protocol, the user may further set the scanning parameters in the imaging protocol.

The user may be a doctor, technician, or other person who may operate the second imaging device before and/or in the scan on the target subject.

In some embodiments, the processing device may send the one or more imaging protocols to the user, and the user may determine one of the imaging protocols.

The processing device may adjust the scanning route in various ways. In some embodiments, in response to determining that the collision is involved in the scan, the processing device may adjust the scanning route by adjusting the rotation angle and adjusting positions of the target subject and/or components of the second imaging device, or the like.

In some embodiments, the processing device may determine whether a collision is involved in the simulated scan.

In some embodiments, the processing device may perform a collision detection based on the 3D spatial model fused with the combination model before the second imaging device performs an actual scanning of the target subject. The simulated scan performed on the 3D geometric model may simulate an actual scan to be performed on the target subject. During the simulated scan, each component in a 3D model representation of the second imaging device may be simulated to move according to a scanning route of a corresponding component of the second imaging device. In some embodiments, the processing device may cause the terminal device to display the 3D spatial model and the processing device may simulate the scan using the 3D spatial model and simulate the movement of the one or more components of the second imaging device according to the scanning route. The processing device may cause the terminal device to display the simulated movement trajectory (i.e., the scanning route) of the one or more components of the second imaging device.

More description of the collision detection may be found in FIG. 2 and related descriptions.

In some embodiments, for the components of the second imaging device, the processing device may determine a distance between the 3D geometric model of the component and the 3D model of other objects (e.g., the target subject) in the simulated scan. The processing device may further determine whether a collision is involved between other objects (e.g., the target subject) and components based on the distance. For example, the processing device may determine whether the distance is less than the distance threshold (e.g., 1 mm, 5 mm, 1 cm). The distance threshold may be manually set by the user or determined by the processing device. In some embodiments, the 3D model of the component may be moved to different positions based on the scanning route of the corresponding component of the second imaging device. The at least two distances between the 3D model of other objects (e.g., the target subject) and the 3D models of the components at different positions may be determined. The processing device may determine whether a collision is involved between the target subject and the components based on each of at least two distances.

In some embodiments, after adjusting the position of the target subject and/or the components of the second imaging device, the processing device may obtain images of the target subject to determine updated position information of the target subject, and/or determine the scanning route for component updated based on the updated position information of the components of the second imaging device. The processing device may scan the target subject based on the updated scanning route.

In some embodiments, the processing device may further determine whether a collision is involved between other objects and one or more components of the second imaging device based on the updated scanning route, and the above process may be repeated until no collision is involved between other objects and the one or more components of the second imaging device.

In some embodiments, by performing the simulated scan based on the scanning route, predicting whether the collision is involved in the scan can improve the accuracy of determining whether the collision is involved in the scan, which can effectively avoid the collision involving between any pair of objects in real-time, thereby avoiding damage to the target object and improving the efficiency of scan.

In some embodiments, in response to determining that the collision is involved in the scan, the processing may generate a reminder for the collision.

The reminder refers to a message related to an event that occurred in the scan. For example, the reminder may include at least one of collision alarm, a position of the collision, or the adjustment of the scanning route.

In some embodiments, in response to determining that the collision is involved in the scan, the processing device may be configured to cause the terminal device to generate a reminder to the user. The reminder may be configured to remind the user of a potential collision. For example, the reminder may be provided to the user in the form of text, voice messages, images, animations, videos, or the like.

In some embodiments, the user may input instructions or information in response to the reminder. For example, the user may manually adjust the target portion of the scanning bed or remove other objects.

In some embodiments of the present disclosure, through the reminder for the collision, the collision alarm, the position of the collision, or the adjustment of the scanning route may be generated, which is conducive to timely obtaining the control instructions for the target object or the second imaging device to avoid the collision.

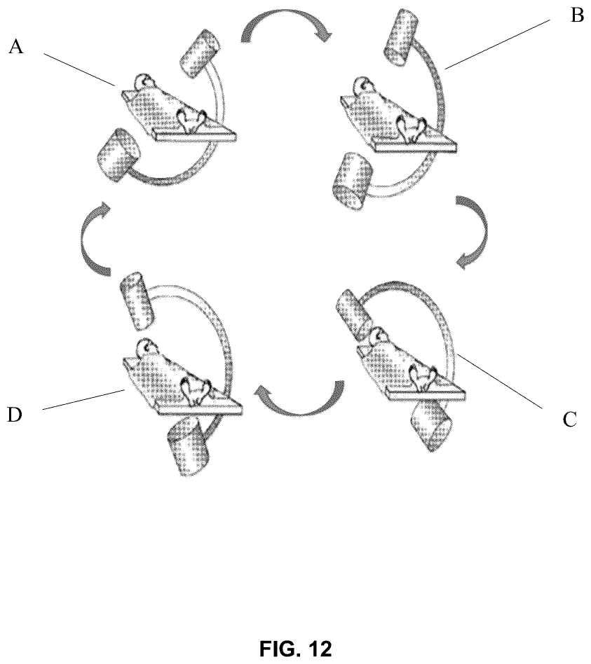

FIG. 12 is a schematic diagram illustrating an exemplary rotating scanning process of a second imaging device according to some embodiments of the present disclosure.

In some embodiments, the process for controlling the scan of a portion of the target subject may further include the following operations.

In response to determining that no collision is involved between the second imaging device and the target object in the space (e.g., a treatment room, a scanning room, etc.) in the simulated scan, the second imaging device may be controlled to scan around the target portion according to an actual scanning route.

In some embodiments, the processing device may automatically identify the color image information and/or depth image information of the target subject through the imaging device (e.g., the camera), and generate a 3D geometric model of the target subject based on the color image information and/or depth image information. The processing device may display the 3D geometric model to the user interface, and combine a portion of the target subject (e.g., a heart model) and the 3D geometric model reconstructed by the camera to obtain a combination model, which can facilitate the user to obtain a target portion of the target subject from the combination model. When a rotation acquisition is performed on the target portion (e.g., a heart), the processing device only needs to select a region of the target portion (e.g., the heart) on the combination model of the target subject, and trigger a one-click in place control (APC). The processing device may automatically move the target portion (e.g., the heart) of the target subject to a rotation center (i.e., the isocenter) of the C-arm. The processing device may correct a rotation angle based on the distance between the second imaging device and the target object in the space (e.g., a treatment room, a scanning room, etc.) (e.g., based on the distance between the second imaging device and the target subject, i.e., the rotation angle may be corrected based on the size of the target subject), and simulate and calculate whether the collision involves among the C-arm/frame and surrounding medical devices or the target subject on the corrected rotation scanning route simultaneously. When determining that no collision is involved in the scan, the processing device may control the C-arm of the second imaging device to automatically rotate and scan around the target portion (e.g., the heart) of the target subject. Automatic positioning of the portion can be realized based on computer vision technology and automatically adjusted the rotation angle based on the size of a portion of the target subject or the distance between the medical device and the second imaging device, the need for the user to repeatedly operate can be eliminated, and the radiation exposure during the operation process and the preparation time for rotation acquisition of the target portion (e.g., the heart) can be reduced. In some embodiments, the imaging device (e.g., the camera) technology may be utilized to combine the application of computer vision and the workflow of the space (e.g., a treatment room, a scanning room, etc.), which can simplify the operation of clinical interventional surgeons, improve the convenience and surgical efficiency during the interventional surgery process.

For example, during the heart rotation acquisition process, the processing device may determine a position of the heart of the patient through the imaging device, and move an isocenter of the second imaging device to the position of the heart. Then, the processing device may perform a simulated scan based on a preset protocol rotation angle, and adjust the rotation angle based on the distance between the second imaging device and the target object in the space (e.g., a treatment room, a scanning room, etc.) calculated in the simulation scan. For example, as scanning positions of the second imaging device shown in FIG. 12, a preset initial rotation angle of A-B-C-D (i.e., the preset initial rotation angle is deviating to the left and to the foot (30° left anterior oblique position, 30° foot oblique position)-deviating to the left and the head (30° left anterior oblique position, 30° head oblique position)-deviating to the right and the head (30° right anterior oblique position, 30° head oblique position)-deviating to the right and the foot (30° right anterior oblique position, 30° foot oblique position). When the distance is relatively small determined through the simulated scan, when the patient is overweight, the angle may be reduced, such as adjusting the angle to 28°; when the distance determined through the simulation scan is relatively large and the patient is thin, the angle may be increased, such as adjusting the angle to 32°. The processing device may also adjust the angle information of a certain scanning position according to the actual situation.

In some embodiments, the rotation angle of the second imaging device may be obtained in the scan and the distance between the second imaging device and the target object in the space (e.g., a treatment room, a scanning room, etc.) by controlling the movement of the second imaging device to move the target portion of the target subject to the isocenter of the second imaging device. Adjusting the rotation angle based on distance may avoid the collision between the second imaging device and the target object in the space (e.g., a treatment room, a scanning room, etc.) by adjusting the rotation angle, thereby improving the acquisition efficiency of the target portion.

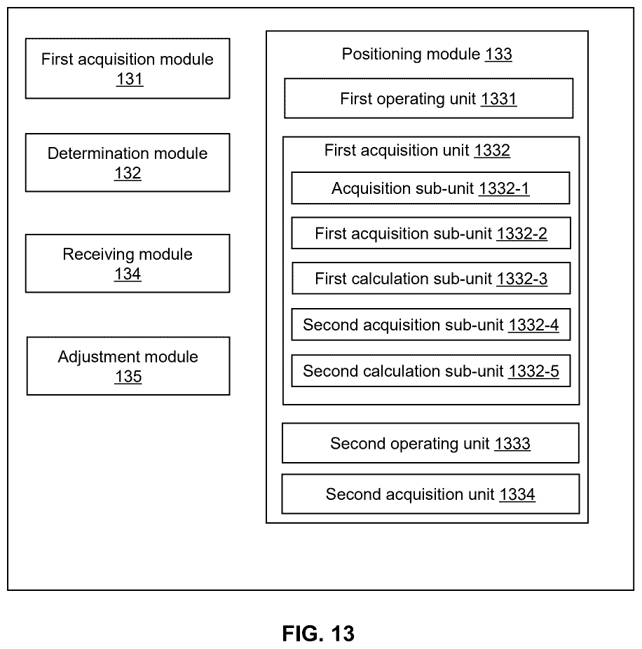

FIG. 13 is a schematic diagram illustrating an exemplary module of a scanning system according to some embodiments of the present disclosure.

The embodiment provides a scanning system suitable for the digital subtraction angiography device as shown in FIG. 13. The system may include an acquisition module 131, a determination module 132, and a positioning module 133;

The first acquisition module 131 may be configured to obtain a target portion of a target subject; more descriptions of obtaining the target portion of the target subject may be found in FIG. 2 and related descriptions.

The determination module 132 may be used to determine a scanning starting ending point position and a scanning ending point position based on the target portion; more descriptions of determining the scanning starting ending point position and the scanning ending point position may be found in FIG. 3 and related descriptions.

The positioning module 133 may be configured to obtain a target image of the target portion by controlling the digital subtraction angiography device to move to the scanning starting ending point position and/or the scanning ending point position. More description of controlling the digital subtraction angiography device to move to the scanning starting ending point position and/or the scanning ending point position may be found in FIG. 3 and related descriptions.

In some embodiments, as shown in FIG. 13, the stepping acquisition system may further include a receiving module 134 and an adjustment module 135;

The receiving module 134 may be configured to receive voice input instructions and/or image input instructions for adjusting the scanning starting ending point position and/or scanning ending point position;

The adjustment module 135 may be configured to obtain the adjusted scanning starting ending point position and/or the adjusted scanning ending point position based on the voice input instructions and/or image input instructions. More descriptions of the adjusted scanning starting ending point position and/or the adjusted scanning ending point position may be found in FIG. 4 and related descriptions.

In some embodiments, as shown in FIG. 13, the positioning module 133 may include a first operating unit 1331;

The first operating unit 1331 may be used to obtain a first image of the target portion by controlling the digital subtraction angiography device to move to the scanning starting ending point position and/or scanning ending point position at a preset speed and acquiring images in the first image acquisition stage before the target portion of the target subject is injected with the contrast agent. More descriptions of obtaining the first image of the target portion may be found in FIG. 3 and related descriptions.

In some embodiments, as shown in FIG. 13, the positioning module 133 may include a first acquisition unit 1332 and a second operating unit 1333;

The first acquisition unit 1332 may be configured to obtain a blood flow velocity of the target portion in the second image acquisition stage after the target portion of the target subject is injected with the contrast agent;

The second operating unit 1333 may be configured to obtain a second image of the target portion by controlling the digital subtraction angiography device to move to the scanning starting ending point position and/or scanning ending point position based on the blood flow velocity of the target portion and acquiring the images. More descriptions of obtaining the second image may be found in FIG. 3 and related descriptions.

In some embodiments, as shown in FIG. 13, the positioning module 133 may include a first operating unit 1331, a first acquisition unit 1332, a second operating unit 1333, and a second acquisition unit 1334;

The first operating unit 1331 may be configured to obtain a first image of the target portion by controlling the digital subtraction angiography device to move to the scanning starting ending point position and/or scanning ending point position at a preset speed and acquiring the images in the first image acquisition stage before the target portion of the target subject is injected with the contrast agent;

The first acquisition unit 1332 is used to obtain the blood flow velocity of the target portion in the second image acquisition stage after the target portion of the target subject is injected with the contrast agent;

The second operating unit 1333 may be configured to obtain a second image of the target portion by controlling the digital subtraction angiography device to move to the scanning starting ending point position and/or the scanning ending point position according to the blood flow velocity of the target portion and acquiring the images;

The second acquisition unit 1334 may be configured to obtain a target image of the target portion based on the first image and the second image. More descriptions of obtaining the target image of the target portion may be found in FIG. 3 and related descriptions.

In some embodiments, as shown in FIG. the first acquisition unit 1332 may include an acquisition sub-unit 1332-1, a first acquisition sub-unit 1332-2, and a first calculation sub-unit 1332-3;

The acquisition sub-unit 1332-1 may be configured to collect a third image of the target portion in the second image acquisition stage after the target portion of the target subject is injected with the contrast agent;

The first acquisition sub-unit 1332-2 may be configured to obtain grayscale values of the third images of the contrast agent at different positions based on the third image;

The first calculation sub-unit 1332-3 may be configured to calculate the blood flow velocity of the target portion based on the grayscale values of the third image of the contrast agent at different positions. More description of obtaining the target image of the target portion may be found in FIG. 3 and related descriptions.

In some embodiments, as shown in FIG. 13, the first acquisition unit 1332 may further include a second acquisition sub-unit 1332-4 and a second calculation sub-unit 1332-5;

The second acquisition sub-unit 1332-4 may be configured to obtain a moving distance of the contrast agent at the target portion and a duration corresponding to the moving distance in the second image acquisition stage after the target portion of the target subject is injected with the contrast agent;

The second calculation sub-unit 1332-5 may be configured to calculate the blood flow velocity of the target portion based on the moving distance and the duration. More description of calculating the blood flow velocity of the target portion based on the moving distance and the duration may be found in FIG. 3 and related descriptions.

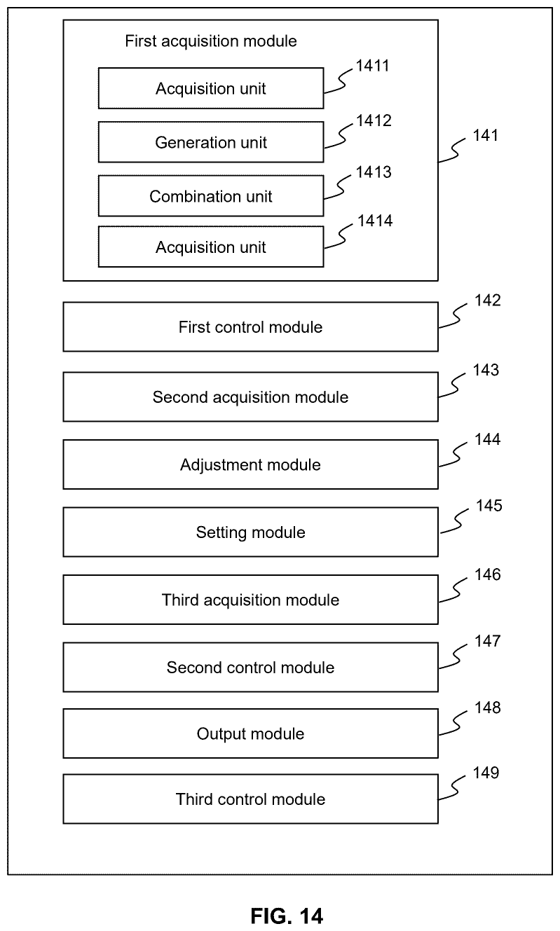

FIG. 14 is a schematic diagram illustrating an exemplary module of a control system for scanning a portion of a target subject according to some embodiments of the present disclosure.

The embodiment provides a control system for scanning a portion of the target subject, as shown in FIG. 14, the control system may include a first acquisition module 141, a first control module 142, a second acquisition module 143, and an adjustment module 144;

The first acquisition module 141 may be configured to obtain a target portion of the target subject; more descriptions of the target portion may be found in FIG. 8 and related descriptions.

The first control module 142 may be configured to control the movement of the second imaging device according to the received control instructions so that the target portion is located at an isocenter point of the second imaging device; the second acquisition module 143 may be configured to obtain a rotation angle of the second imaging device in the scan and a distance between the second imaging device and the target object in the space (e.g., a treatment room, a scanning room, etc.); more descriptions of locating the target portion at the isocenter point of the second imaging device may be found in FIG. 8 and related descriptions. More descriptions of obtaining the rotation angle of the second imaging device and the distance between the second imaging device and the target object in the space (e.g., a treatment room, a scanning room, etc.) may be found in FIG. 8 and related descriptions.

The adjustment module 144 may be configured to adjust the rotation angle based on the distance. More description of adjusting the rotation angle may be found in FIG. 8 and related descriptions.

In some embodiments, as shown in FIG. 14, the first acquisition module 141 may include an acquisition unit 1411, a generation unit 1412, a combination unit 1413, and an acquisition unit 1414.

The acquisition unit 1411 may be configured to acquire color image information and/or depth image information of the target subject; more description of acquiring the color image information and/or depth image information of the target subject may be found in FIG. 9 and related descriptions.

The generation unit 1412 may be configured to generate a 3D geometric model of the target subject based on the color image information and/or the depth image information, more descriptions may be found in FIG. 9 and related descriptions.

The combination unit 1413 may be configured to obtain a combination model by combining the anatomical structure model of the target subject and the 3D geometric model, more descriptions may be found in FIG. 9 and related descriptions.

The acquisition unit 1414 may be configured to obtain a target portion of the target subject from the combination model; more descriptions of obtaining the target portion of the target subject from the combination model may be found in FIG. 9 and related descriptions.

In some embodiments, in response to determining that the distance is less than or equal to a distance threshold, the adjustment module 144 may be configured to adjust a rotation angle, such that a distance between the second imaging device after adjusting the rotation angle and the target object in the space (e.g., a treatment room, a scanning room, etc.) may be greater than the distance threshold. More descriptions of adjusting the rotation angle may be found in FIG. 9 and related descriptions.

In some embodiments, as shown in FIG. 14, the control system may further include a setting module 145;

The setting module 145 may be configured to set an adjustment amplitude of the rotation angle based on the distance;

In response to determining that the distance is less than or equal to a distance threshold, the adjustment module 144 may be configured to adjust a rotation angle according to the adjustment amplitude, such that a distance between the second imaging device after adjusting the rotation angle and the target object in the space (e.g., a treatment room, a scanning room, etc.) may be greater than the distance threshold. More descriptions of adjusting the rotation angle according to the adjustment amplitude may be found in FIG. 10 and related descriptions.

In some embodiments, as shown in FIG. 14, the control system may further include a third acquisition module 146, a second control module 147, and an output module 148;

The third acquisition module 146 may be configured to obtain a preset scanning route;

The second control module 147 may be configured to scan around the target portion by simulating the second imaging device according to a preset scanning route;

In response to determining that a collision is involved between the second imaging device and the target object in the space (e.g., a treatment room, a scanning room, etc.) in the simulated scan, the output module 148 may be configured to output collision alarm information, position information of the collision, and rotation angle adjustment information. More descriptions of the simulated scan, outputting the collision alarm information, the position information of the collision, and the rotation angle adjustment information may be found in FIG. 11 and related descriptions.

In some embodiments, as shown in FIG. 14, the control system may further include a third control module 149;

In response to determining that no collision involves between the second imaging device and the target object in the space (e.g., a treatment room, a scanning room, etc.) in the simulated scan, the third control module 149 may be configured to control the second imaging device to perform a scan around the target portion according to an actual scanning route.

More descriptions of determining that whether the collision involves between the second imaging device and the target object in the space (e.g., a treatment room, a scanning room, etc.) in the simulated scan may be found in FIG. 11 and related descriptions.

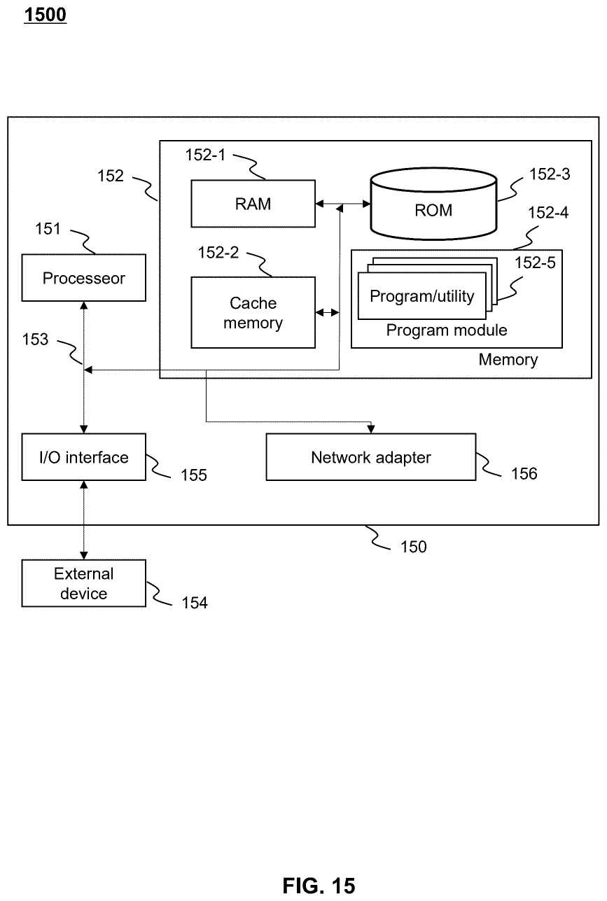

FIG. 15 is a schematic diagram illustrating an exemplary structure of an electronic device according to some embodiments of the present disclosure.

The electronic device may include a memory, a processor, and a computer program stored in the memory that may operate on the processor. When the processor executes the program, the control method for scanning h a portion of the target subject as described in the above embodiments. The electronic device displayed in FIG. 15 may be merely an example, which may not be limited to functions and using the scope of the embodiments in the present disclosure.

As shown in FIG. 15, the electronic device 1500 may be represented in a form of a universal computing device, such as a server device. The components of the electronic device 1500 may include but are not limited to at least one processor 151, at least one memory 152, and buses 153 connecting different system components (including the memory 152 and the processor 151).

The buses 153 may include a data bus, an address bus, and a control bus.

The memory 152 may include a volatile memory, such as random access memory (RAM) 152-1 and/or a cache memory 152-2, and may further include a read-only memory (ROM) 152-3.

The Memory 152 may also include a program/utility 152-5 with a set (or at least one) of program module 152-4, such a program module 152-4 including but not limited to: an operating system, one or more application programs, other program modules, and program data, each of which may include an implementation of a network environment.

The processor 151 may execute various functional applications and data processing by running computer programs stored in the memory 152, such as a control method for scanning a portion of a target subject described in above embodiments.

The electronic device 1500 may also communicate with one or more external devices 154 (e.g., keyboards, pointing devices, etc.). The communication may be carried out through an input/output (I/O) interface 155. Moreover, the electronic device 1500 generated by the model may communicate with one or more networks (e.g., a local area network (LAN), a wide area network (WAN), and/or a public network (e.g., Internet) through a network adapter 156. As shown in FIG. 15, THE network adapter 156 may communicate with other modules of the electronic device 1500 generated by the model through the buses 153. It should be understood that although not shown in the figure, other hardware and/or software modules may be used in conjunction with the electronic device 1500 generated by the model, including but not limited to: a microcode, a device driver, a redundant processor, an external disk drive array, a disk array (RAID) system, a tape drive, a data backup storage system, or the like.

It should be noted that although several units/modules or subunits/modules of the electronic devices are mentioned in the detailed description above, the division is only exemplary and not mandatory. In fact, according to the embodiments of the present disclosure, features and functions of two or more units/modules described above may be embodied in one unit/module. On the contrary, the features and functions of the unit/module described above may be further divided into multiple units/modules to be concretized.

A method may be provided in one or more embodiments of the present disclosure, the method may be implemented on a computing apparatus, the computing apparatus may include at least one processor and at least one storage device, comprising: obtaining one or more images of a target subject acquired by an imaging device; determining a three-dimensional (3D) geometric model of the target subject based on the one or more images; obtaining an anatomical structure model of at least a portion of the target subject; obtaining a combination model by combining the 3D geometric model of the target subject and the anatomical structure model of at least a portion of the target subject; and determining one or more scanning parameters of the target subject based on the combination model.

A non-transitory computer readable storage medium may be provided in one or more embodiments of the present disclosure, the storage medium may store computer instructions, wherein after the computer reads the computer instructions in the storage medium, the computer may perform the scanning method described in above embodiments.

The readable storage medium may include, but is not limited to, a portable disk, a hard drive, a random-access memory, a read-only memory, an erasable programmable read-only memory, an optical storage device, a magnetic memory device, or any combination thereof.

In some embodiments, the present disclosure may also be implemented in the form of a program product, which may include program codes. When the program product is operated on the terminal device, the program codes may be used to cause the terminal device to execute the stepping acquisition method described in the embodiment described above.

The program codes for executing the present disclosure may be written in any combination of one or more programming languages. The program codes may be completely executed on the user device, partially executed on the user device, executed as a standalone software package, partially executed on the user device, partially executed on the remote device, or completely executed on the remote device.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (Saas).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A system, comprising:
at least one storage medium including a set of instructions;
at least one processor in communication with the at least one storage medium, wherein when executing the set of instructions, the at least one processor is directed to cause the system to perform operations including:
obtaining one or more images of a target subject acquired by an imaging device;
determining a three-dimensional (3D) geometric model of the target subject based on the one or more images;
obtaining an anatomical structure model of at least a portion of the target subject;
obtaining a combination model by combining the 3D geometric model of the target subject and the anatomical structure model of at least a portion of the target subject; and
determining one or more scanning parameters of the target subject based on the combination model.

2. The system of claim 1, wherein the determining one or more scanning parameters of the target subject based on the combination model includes:
displaying the combination model on a user interface;
obtaining a first input of a user via the user interface generated according to the combination model; and
determining the one or more scanning parameters based on the first input of the user.

3. The system of claim 1, wherein the operations further include:
obtaining a second input of the user via the user interface generated according to the combination model; and
adjusting the one or more scanning parameters based on the second input of the user.

4. The system of claim 1, wherein the one or more scanning parameters include a scanning range defined by a starting point and an ending point, the operations further include:
in a first image acquisition stage before a target portion of the target subject is injected with a contrast agent, causing a second imaging device to arrive at the starting point and/or the ending point; and
obtaining a first image by causing the second imaging device to perform a scan.

5. The system of claim 1, wherein the operations further include:
in a second image acquisition stage after the target portion of the target subject is injected with the contrast agent, determining a blood flow velocity of the target portion;
adjusting a moving speed of the second imaging device based on the blood flow velocity; and
obtaining a second image by causing the second imaging device to perform a scan.

6. The system of claim 5, wherein the operations further include:
obtaining a target image of the target portion of the target subject based on the first image and the second image.

7. The system of claim 5, wherein the determining a blood flow velocity of the target portion includes:
obtaining a third image of the target portion of the target subject; and
determining the blood flow velocity based on the third image.

8. The system of claim 5, wherein the determining a blood flow velocity of the target portion includes:
determining a moving distance and a duration corresponding to the moving distance of the contrast agent in the target subject; and
determining the blood flow velocity based on the moving distance and the duration corresponding to the moving distance of the contrast agent in the target subject.

9. The system of claim 1, further comprising:
causing a second imaging device to perform multiple rounds of scans on the target portion of the target subject based on the one or more scanning parameters.

10. The system of claim 1, wherein the target portion includes the heart of the target subject, and the causing a second imaging device to perform multiple rounds of scans on the target portion of the target subject based on the one or more scanning parameters includes:
causing the second imaging device to perform a rotation scan on the heart, the rotation scan including the multiple rounds of scans, each of the multiple rounds of scans corresponding to a rotation angle.

11. The system of claim 1, wherein the target portion includes a leg of the target subject, and the causing a second imaging device to perform multiple rounds of scans on the target portion of the target subject based on the one or more scanning parameters includes:
causing the second imaging device to perform a stepping scan on the leg, the stepping scan including the multiple rounds of scans, each of the multiple rounds of scans corresponding to one bed position.

12. (canceled)

13. The system of claim 1, wherein the anatomical structure model of the at least a portion of the target subject is acquired based on one or more images acquired by a second imaging device.

14. The system of claim 1, wherein the one or more scanning parameters include a scanning range, the operations further include:
causing one or more components of a second imaging device to move to a target position, at the target position, the scanning range of the target subject being located at an isocenter of the second imaging device; and
causing the second imaging device to perform a scan on the target subject.

15. The system of claim 1, wherein the one or more scanning parameters include a rotation angle of one or more components of a second imaging device for performing a scan on the target subject, and the determining one or more scanning parameters of the target subject based on the combination model includes:
determining the rotation angle by adjusting an initial rotation angle.

16. The system of claim 15, wherein the adjusting an initial rotation angle includes:
determining a distance between the target subject and the one or more components of the second imaging device based on the combination model; and
adjusting the initial rotation angle based on the distance.

17. The system of claim 16, wherein the adjusting the initial rotation angle based on the distance includes:
in response to determining that the distance is less than a distance threshold, adjusting the initial rotation angle to obtain the rotation angle, a distance between the target subject and the one or more components of the second imaging device after the initial rotation angle is adjusted exceeds the distance threshold.

18. The system of claim 1, wherein the one or more scanning parameters includes a scanning route of one or more components of a second imaging device for performing a scan on the target subject, the scanning route indicating a moving trajectory of the one or more components of the second imaging device, and the operations further include:
predicting whether a collision involves in the scan based on the scanning route;
in response to determining that the collision involves in the scan, adjusting the scanning route; and
causing the second imaging device to perform the scan based on the adjusted scanning route.

19. (canceled)

20. The system of claim 1, wherein the determining one or more scanning parameters of the target subject based on the combination model includes:
obtaining a trained machine learning model; and
determining the one or more scanning parameters of the target subject based on the combination model and the trained machine learning model.

21. A method implemented on a computing apparatus, the computing apparatus including at least one processor and at least one storage device, comprising:
obtaining one or more images of a target subject acquired by an imaging device;
determining a three-dimensional (3D) geometric model of the target subject based on the one or more images;
obtaining an anatomical structure model of at least a portion of the target subject;
obtaining a combination model by combining the 3D geometric model of the target subject and the anatomical structure model of at least a portion of the target subject; and
determining one or more scanning parameters of the target subject based on the combination model.

22. A non-transitory computer readable medium, comprising a set of instructions, wherein when executed by at least one processor, the set of instructions direct the at least one processor to perform acts of:
obtaining one or more images of a target subject acquired by an imaging device;
determining a three-dimensional (3D) geometric model of the target subject based on the one or more images;
obtaining an anatomical structure model of at least a portion of the target subject;
obtaining a combination model by combining the 3D geometric model of the target subject and the anatomical structure model of at least a portion of the target subject; and
determining one or more scanning parameters of the target subject based on the combination model.
